(19) United States
(12) Patent Application Publication
(10) Pub. No.: US 2024/0052313 A1
(43) Pub. Date: Feb. 15, 2024

(54) CHONDROGENIC HUMAN MESENCHYMAL STEM CELL (MSC) SHEETS

(71) Applicant: UNIVERSITY OF UTAH RESEARCH FOUNDATION, Salt Lake City, UT (US)

(72) Inventors: Teruo Okano, Salt Lake City, UT (US); Kyungsook Kim, Murray, UT (US); Hallie Thorp, Salt Lake City, UT (US); David W. Grainger, Salt Lake City, UT (US); Travis Maak, Park City, UT (US)

(73) Assignee: UNIVERSITY OF UTAH RESEARCH FOUNDATION, Salt Lake City, UT (US)

(21) Appl. No.: 17/766,292

(22) PCT Filed: Oct. 7, 2020
(86) PCT No.: PCT/US2020/054477

§ 371 Date: Apr. 4, 2022

Related U.S. Application Data
(60) Provisional application No. 62/911,441, filed on Oct. 7, 2019.

(51) Int. Cl. C12N 5/077; A61P 19/02

(52) U.S. Cl. CPC C12N 5/0655; A61P 19/02; C12N 2506/1353; C12N 2533/54; C12N 2501/155; C12N 2501/60; C12N 2501/998; C12N 2501/15

(57) ABSTRACT

The disclosure relates to a chondrogenic cell sheet comprising at least two layers of confluent chondrogenically differentiated cells, wherein the cell sheet is prepared from mesenchymal stem cells (MSCs), and wherein the chondrogenically differentiated cells on the basal side of the cell sheet express adhesion molecules. The disclosure also relates to a method of preparing a chondrogenic cell sheet comprising: a) culturing MSCs on temperature responsive cultureware until confluent to form a cell sheet; b) detaching the cell sheet by temperature reduction and allowing the cell sheet to contract, forming a contracted cell sheet; c) contacting the contracted cell sheet with a culture surface; and d) treating the contracted cell sheet on the culture surface with chondrogenic medium and culturing to form a chondrogenic cell sheet. Methods of using the chondrogenic cell sheets to repair cartilage tissue or treat joint disease are also disclosed.

### RELATED APPLICATIONS

This application claims priority to U.S. Provisional Patent Application No. 62/911,441 filed on Oct. 7, 2019, the contents of which are incorporated herein in their entirety.

### BACKGROUND

Degenerative joint disease, such as osteoarthritis (OA), is increasingly responsible for morbidity and compromising quality of life in the global population. OA is a systemic and poorly understood disease, making it challenging to treat successfully once degeneration has begun. Traumatic injuries, often to the knee joint, can result in focal defects in the articular cartilage, and these articular cartilage focal defects are responsible for more than 12% of cases of OA. Lacking a direct blood supply, articular cartilage has minimal ability to spontaneously repair; therefore, cartilage injuries are rarely able to heal without intervention. Healthy articular cartilage exhibits a hyaline structure, and the goal for focal defect therapies is promotion of cartilage regeneration toward a hyaline-like phenotype at the site of injury. Bone marrow stimulation techniques, such as microfracture, are the most frequently used method in clinical practice; however, resulting mixed fibrocartilage tissue is inferior to native hyaline cartilage, diminishing functionality of the cartilage. To properly treat OA and decrease the incidence of this systemic and debilitating disease, improved treatment options that regenerate hyaline cartilage at the site of articular cartilage focal defects before they progress to OA must be developed.

In an attempt to improve outcomes for articular cartilage focal defect therapies, autologous chondrocyte implantation was developed. These approaches rely on autologous cell sourcing, where the patient is both the donor and the recipient, which increases patient burden with multiple surgeries, donor-dependent cell quality, and lengthy cell expansion and preparation time. Additionally, these therapies use cell suspensions or seeded scaffolds to deliver the cells, which offer poor localization to the target tissue site and poor retention of cell functionality. Despite promising results brought about by advances in tissue engineering, various limitations remain unsolved, including a lengthy cell expansion period (large-scale production; off-the-shelf product) and the cell delivery system (low transplantation efficacy)

Growing efforts are transitioning from autologous chondrocyte sources to “off-the-shelf” allogeneic cell sourcing. Mesenchymal stem cells (MSC) as an allogeneic cell source offer well documented regenerative and immunomodulatory properties, as well as standards for preparing cells with specific phenotypes. MSC multipotency, including chondrogenic lineages, enables directed differentiation to chondrocyte phenotypes in vitro within 3D constructs, usually pellet cultures. In vitro chondrogenic differentiation of MSCs is well-documented; however, for cells to be transplanted in vivo, these cells are either digested enzymatically and delivered as a cell suspension or seeded into a scaffold material. The cells' chondrogenic potential is rarely realized after transplantation because the cellular environments are altered during enzymatic cell harvest and during interfacing with the scaffold construct, and as such, MSC applications for cartilage focal defect healing fail before reaching the clinic. Even though there are many advantages to using MSCs for cartilage regeneration, there is no method for preparing a construct that has differentiation potential and allows transplantation of in vitro differentiated hyaline cartilage without disrupting the structural or chondrogenic characteristics. One of the major hurdles to translation of MSC cell therapies is the ability to reliably transplant and maintain localization of administered cells. Thus a need exists for developing chondrogenic cell sheets with hyaline-like characteristics that may be successfully transplanted to host tissue.

### SUMMARY OF THE INVENTION

In certain aspects the disclosure relates to a chondrogenic cell sheet comprising at least two layers of confluent chondrogenically differentiated cells, wherein the cell sheet is prepared from mesenchymal stem cells (MSCs), and wherein the chondrogenically differentiated cells on the basal side of the cell sheet express one or more adhesion molecules. In certain embodiments, the MSCs are human bone marrow MSCs (hBM-MSCs). In certain embodiments, the cell sheet consists essentially of chondrogenically differentiated cells. In certain embodiments, at least 50% of cells in the cell sheet are chondrogenically differentiated cells. In certain embodiments, the cell sheet comprises an extracellular matrix.

In certain embodiments, the extracellular matrix comprises a protein selected from the group consisting of type II collagen and a sulphated proteoglycan. In certain embodiments, the chondrogenically differentiated cells express a protein selected from the group consisting of SOX9, aggrecan, COL2A1, ACAN, COMP and BMP2. In certain embodiments, the cell sheet comprises lacunae structures. In certain embodiments, the one or more adhesion molecules are selected from fibronectin and laminin. In certain embodiments, the cell sheet exhibits physical adhesion to a cartilage surface after transplantation to the cartilage surface.

In certain aspects the disclosure relates to a method of preparing a chondrogenic cell sheet comprising: a) culturing mesenchymal stem cells (MSCs) on temperature responsive cultureware until confluent to form a cell sheet; b) detaching the cell sheet by temperature reduction and allowing the cell sheet to contract, forming a contracted cell sheet; c) contacting the contracted cell sheet with a culture surface; and d) treating the contracted cell sheet on the culture surface with chondrogenic medium and culturing to form a chondrogenic cell sheet.

In certain embodiments, the contracted cell sheet is grown on the culture surface for at least 3 weeks. In certain embodiments, the chondrogenic medium comprises a protein selected from the group consisting of transforming growth factor beta (TGFβ) and a bone morphogenic protein (BMP). In certain embodiments, the cell sheet in step b) is detached from the temperature responsive cultureware without treating the cell sheet with one or more enzymes.

In certain aspects the disclosure relates to a chondrogenic cell sheet produced by the methods described herein.

In certain aspects the disclosure relates to a method of transplanting a chondrogenic cell sheet to a subject in need thereof, comprising applying a chondrogenic cell sheet as described herein to a tissue of a subject. In certain embodiments, the tissue is selected from cartilage and bone. In certain embodiments, the cartilage is articular cartilage.

In certain aspects the disclosure relates to a method of repairing cartilage tissue in a subject in need thereof, comprising applying a chondrogenic cell sheet as described herein to cartilage of the subject, thereby repairing the cartilage tissue in the subject. In certain embodiments, the cartilage is articular cartilage. In certain embodiments, the subject has a focal cartilage defect.

In certain embodiments, the subject has a symptomatic cartilage defect. In certain embodiments, the symptomatic cartilage defect is caused by acute or repetitive trauma. In certain embodiments, the subject has a degenerative joint disease.

In certain aspects the disclosure relates to a method of treating or preventing a joint disease in a subject in need thereof, comprising applying a chondrogenic cell sheet as described herein to a joint in the subject, thereby treating or preventing the joint disease in the subject. In certain embodiments, the joint is selected from the group consisting of a synovial joint and a cartilaginous joint. In certain embodiments, the synovial joint is a knee joint, wrist joint, shoulder joint, hip joint, elbow joint, or neck joint. In certain embodiments, the joint disease is a degenerative joint disease. In certain embodiments, the joint disease is selected from the group consisting of joint inflammation, osteoarthritis, rheumatoid arthritis, and chondromalacia patellae.

In certain aspects the disclosure relates to a method of preventing osteoarthritis in a subject having a symptomatic cartilage defect caused by acute or repetitive trauma, the method comprising applying a chondrogenic cell sheet as described herein to the cartilage having the defect, thereby preventing the osteoarthritis in the subject. In certain embodiments, the subject is human. In certain embodiments, the chondrogenically differentiated cells in the cell sheet are allogeneic to the subject. In certain embodiments, the chondrogenically differentiated cells in the cell sheet are autologous to the subject.

### BRIEF DESCRIPTION OF THE FIGURES

FIG. 1A-1F shows that cell sheet contraction promotes structural rearrangements. Representative macroscopic images of full cell sheets non-contracted (A) and contracted (B) in 35 mm culture dishes. Cell sheet edges marked by dotted orange line. Scale bars=5 mm. Representative cross-sectional histology of H&E for non-contracted (C) and contracted (D) cell sheets. Cell sheet diameter (E) and thickness (F) for contracted compared to non-contracted sheets. Error bars represent means±SD (n=4: *p<0.05, **p<0.01).

FIG. 2A-2I shows differentiation potential of hBM-MSCs at 3 weeks. Representative images of histological sections of pellets in control medium (A-C) and chondrogenic medium (D-F). Stains were Alcian blue for acidic mucins (A,D), Safranin-O/Fast green for sulphated proteoglycans (B,E), and collagen type II (pseudo red) with DAPI (blue) (C,F). Scale bars: 200 μm. Chondrogenic gene expression (G-I) with RT-PCR for Sox9 (G), Collagen Type II (H), and Aggrecan (I). All gene expression normalized to GAPDH and compared to the control samples. Error bars represent means±SD (n=4: *p<0.05, **p<0.01).

FIG. 3 shows chondrogenic differentiation of hBM-MSC monolayers, contracted sheets, and pellets at 3 weeks, marked by histology, IHC, and RT-PCR. (Scale bars=200 μm, *p<0.05, **p<0.01).

FIG. 4 shows healthy Articular Cartilage: Hyaline Cartilage Phenotypes. The goal for cartilage focal defect therapies is replacing or regenerating hyaline-like tissue in vivo. See Castagnola, P. et al. JCB (1986); Moore, D. et al. Ortho B. (2019); and Akkiraju, H. et al. J Dev Biol. (2016).

FIG. 5 shows cell sheets as a cell delivery method. Limitations of cell delivery methods (suspensions, scaffolds, pellets) include poor retention of cells and poor interfacing with the native cartilage tissue. Current tools for chondrogenic differentiation do not achieve complete differentiation to hyaline-like cartilage phenotypes in vitro, while also being directly transplantable to the target tissue site.

FIG. 6 shows advantages of MSC cell sheets. By using cell sheet technology with MSCs it is possible to create a fully scaffold-free 3D construct that allows differentiation to hyaline-like phenotypes during in vitro culture with chondrogenic medium, and a chondrogenic construct that is able to be transplanted directly to the target tissue site without disrupting the structural characteristics or hyaline-like phenotypes.

FIG. 7 shows chondrogenic differentiation of cell sheets. hBM-MSCs exhibit hyaline-like chondrogenic potential as a contracted sheet but not as a non-contracted sheet.

FIG. 8 shows control of cell sheet thickness with layering. In addition to contraction, layering can control thickness and three-dimensionality of the cell sheet construct.

FIG. 9 shows in vitro engineering of transplantable, scaffold-free, 3D hyaline-like cartilage tissue from human bone marrow-derived mesenchymal stem cell (hBM-MSC) sheets. This approach uses cell sheet tissue engineering to prepare scaffold-free 3D MSC cellular constructs via spontaneous post-detachment cell sheet contraction from temperature-responsive culture dishes (TRCD). This technology allows in vitro differentiation to hyaline-like cartilage phenotypes by re-plating 3D contracted sheets to cell culture inserts and inducing with chondrogenic media for 3 weeks. Direct transplantation of sheets to target tissue post-differentiation is also possible without damaging the structure or chondrogenic characteristics of the sheet construct.

FIG. 10A-10F shows that cell sheet contraction promotes cytoskeletal rearrangements and pro-chondrogenic signaling compared to 2D cell culture. Representative confocal images of full dish (top-down) (a) 2D cultures and (b) 3D contracted sheets before chondrogenic induction at day-0 with phalloidin/F-actin (green) staining for cytoskeleton and DAPI (blue) nuclear staining. Quantitative real-time PCR gene expression for (c) cytoskeletal structure marker β-actin, (d) cell-cell marker β-catenin, and chondrogenic ECM markers (d) BMP2 and (f) COMP. All gene expression normalized to GAPDH and compared to the 2D cell culture 0-day control sample. Error bars represent means±SD (n=4) (*p<0.05, **p<0.01).

FIG. 11A-11T shows that cell sheet contraction stimulates enhanced chondrogenic differentiation of hBM-MSCs. Comparison between 3-week chondrogenically induced hBM-MSC sheets before (2D culture) and after (3D cell sheet) post-detachment sheet contraction. Representative images of histological cross-sections of hBM-MSC constructs in (a-f) control medium and (g-l) chondrogenic medium for 3 weeks. Stains were (a,b,g,h) Safranin-O/Fast green for sulfated proteoglycans, (c,d,i,j) type II collagen (pseudo red) with DAPI (blue), and (e,f,k,l) type I collagen (red) with DAPI (blue). Representative full dish (top-down) confocal images of 3-week chondrogenically-induced (m) 2D cultures and (n) 3D cell sheets with phalloidin/F-actin (green) staining for cytoskeleton and DAPI (blue) nuclear staining. Cell sheet (o) thicknesses and (p) linear nuclei density for 2D cultures and 3D cell sheets at 0-day (grey bar) and 3-week (black bar) differentiation. Chondrogenic gene expression with quantitative real-time PCR for (q) SOX9, (r) aggrecan, and (s) type II collagen. (t) Ratios of type II to type I collagen shown as ratios of relative gene expression. All gene expression normalized to GAPDH and compared to the 2D culture 3-week control samples. Error bars represent means±SD (n=4) (ns: p≥0.05, *p<0.05, **p<0.01).

FIG. 12A-12U shows progression of chondrogenic differentiation over time for 3D hBM-MSC cell sheets compared to standard pellet cultures. Representative images of histological cross-sections of (a-d, i-l) pellets and (e-h, m-p) 3D cell sheets in chondrogenic medium for 0 days −3 weeks stained with (a-h) Safranin-O/Fast green for sulfated proteoglycans and (i-p) MMP13 (green) with DAPI (blue). Representative images of histological cross-sections of pellets and 3D cell sheets at 3 weeks in chondrogenic media stained for (q) type II collagen (pseudo red) and (r) type I collagen (red) with DAPI (blue). Quantitative real-time PCR of pellets (dashed blue) and 3D cell sheets (solid black) for (s) chondrogenic gene expression: SOX9, type II collagen, and aggrecan; (t) hypertrophic and fibrocartilage gene expression: type X collagen and MMP13; (u) cell-cell interaction expression: β-catenin. All gene expression normalized to GAPDH and compared to single cell 0-day control samples. Error bars represent means±SD (n=4). (*p<0.05, **p<0.01).

FIG. 13A-13F shows manipulation and secondary adhesion capabilities of 3D contracted hBM-MSC cell sheets and pellet cultures post-differentiation to FBS coated surfaces. Representative images of Safranin-O stained histological cross-sections of 3-week differentiated 3D sheets (a) before transfer and (b) 3 days after transfer to an FBS-coated surface. (c) Representative phase-contrast images of sheet edges from 0-72 hours after transfer. Cell migration from cell sheet edges marked by dotted orange line. (d) Attachment efficacy of 3-week differentiated pellets and 3D cell sheets after 1 hour of attachment to secondary FBS-coated culture dishes and 6 hours continued culture (n=6). Construct attachment=((number of attached constructs)/(number of attempted transferred constructs))*100. Representative IHC cross-sectional images of 3-week differentiated (e) contracted sheets and (f) pellet cultures for expression and localization of adhesion molecule laminin with DAPI nuclear stain. Adhesion surfaces ((e) Basal side of the cell sheet and (f) periphery of the pellet culture) marked by dotted yellow lines.

FIG. 14A-14D shows transplantation characteristics for 3D contracted hBM-MSC cell sheets post-differentiation to fresh ex vivo human cartilage pieces. (a) Cell sheets after 3-week differentiation in 35 mm culture dish (b) transplanted onto fresh ex vivo human articular cartilage samples. Transferred cell sheet quadrant marked by dotted orange line. Representative cross-sectional histological and IHC staining of 3-week chondrogenic 3D hBM-MSC sheets (CS) naturally adhered to fresh ex vivo human articular cartilage (hAC) for (c) Safranin-O/Fast-green and (d) cell adhesion molecule laminin counterstained with DAPI.

FIG. 15A-15F shows the transplantation ability of chondrogenically differentiated cell sheets in vivo. Macroscopic images of 2 mm focal defect in nude (RNU) rat hind leg trochlear groove (A) with transplanted 3-week chondrogenically differentiated hyaline-like human MSC-derived cell sheet (B). Cell sheet was transplanted at the time of defect creation. 2 weeks post-transplantation, the knee joint was harvested (C) to assess cell sheet transplantation capacity. Representative cross-sectional histological analysis with Safranin-O (D), Hematoxylin & Eosin (E), and human-specific vimentin immunohistochemical staining (F) of the 3-week differentiated human cell sheet in the rat focal defect 2 weeks post-transplantation. Positive Safranin-O staining (red color) shows retention of positive hyaline-like characteristics of the cell sheet in the transplant area (D). H&E staining indicates integration with the host tissue (E). Transplanted human cells within the cell sheet are positive for human-specific vimentin indicating engraftment and retention in the transplant area (F). Scale bars=200 mm.

### DETAILED DESCRIPTION

This disclosure describes the preparation of mesenchymal stem cells as cell sheets on temperature-responsive cultureware and demonstrates the cell sheets' ability to differentiate to hyaline-like cartilage phenotypes in vitro. The disclosure also demonstrates the cell sheets' transplantation ability to the target tissue site without damaging the structural or chondrogenic characteristics of the construct.

As a next-generation cell delivery method, cell sheet tissue engineering uses temperature-responsive cultureware to produce scaffold-free multi-cell constructs. Regenerative cells are harvested as intact sheets with reproducible physiologic properties and scalable production methods. Cell sheets retain endogenous cell matrix, receptors, and adhesive proteins, enhancing cell viability and communication, retaining endogenous cellular environments, and permitting spontaneous adhesion to biomaterials and biologic surfaces without suturing. Sheet manipulation and layering post-harvest are also possible without compromising cell structure or function.

The chondrogenic cell sheets described herein were demonstrated to transfer onto human cartilage and attach on the cartilage surface with cell adhesion proteins (e.g. laminin) aligned in the interface between cell sheet and cartilage. From this finding, we can expect that the differentiated cell sheet can be transplanted to target tissue easily without any structural changes or damages by the advantage of cell sheet which enables harvesting of cells without any enzyme and retains cell adhesion proteins.

The chondrogenic cell sheets described herein are prepared by culturing mesenchymal stem cells (MSCs) on temperature responsive cultureware until confluent to form a monolayer cell sheet; detaching the cell sheet by temperature reduction to form a contracted cell sheet with multiple layers of cells; contacting the contracted cell sheet with a culture surface; and treating the contracted cell sheet on the culture surface with chondrogenic medium. The chondrogenic cell sheets described herein are three-dimensional, which is important for chondrogenic differentiation. The term “three-dimensional cell sheet” or “3D” cell sheet as used herein refers to a chondrogenic cell sheet that comprises multiple layers of cells and has a reorganized actin cytoskeleton relative to the monolayer cell sheet (e.g. an actin cytoskeleton that is less aligned because it is no longer in tension as it is in monolayer culture on a cell support), and/or has cells/nuclei that are more rounded and less elongated compared to the monolayer cell sheet. In addition, the cell sheet inhibits hypertrophy due to their stable environment. It was possible to transplant the differentiated cell sheet to a target site (i.e. cartilage) because of cell adhesion proteins retained in the cell sheet.

The significant chondrogenic capacity of human bone marrow MSCs (hBM-MSCs) as 3D cell sheets provides a strong foundation for developing a scaffold-free 3D chondrogenic construct that may be used, for example, for articular cartilage focal defect therapies. Cytoskeletal rearrangement after cell sheet contraction is a possible mechanism of increased chondrogenesis. Further tailoring of 3D cell sheet structures for chondrogenesis is possible via sheet layering. This disclosure demonstrates successful engraftment of differentiated 3D chondrogenic cell sheets to ex vivo cartilage, while maintaining positive chondrogenic characteristics. In addition, this disclosure demonstrates that chondrogenically differentiated hyaline-like human MSC-derived cell sheets may be successfully transplanted to cartilage in vivo and integrated with host tissue while maintaining hyaline-like characteristics.

I. Mesenchymal Stem Cells (MSCs)

Mesenchymal stem cells (MSCs) have a remarkable clinical potential to treat a wide range of debilitating diseases, mainly due to their unique immunomodulatory role and regenerative capacity (Caplan and Sorrell, 2015, Immunol Lett 168(2): 136-139). MSCs suitable for use in the methods described herein include, but are not limited to MSCs from bone marrow, umbilical cord, cord blood, limb bud, dental tissue (e.g. molars), adipose tissue, muscle and amniotic fluid.

Any MSCs with chondrogenic potential may be used.

In a particular embodiment, the mesenchymal stem cell is a human bone marrow mesenchymal stem cell (hBM-MSC). The term “human bone marrow mesenchymal stem cell” or “hBM-MSC” as used herein refers to a mesenchymal stem cell that has been isolated from human bone marrow. Methods of isolating hBM-MSCs are known in the art and are described for example, in Baghaei et al., 2017, Gastroenterol Hepatol Bed Bench. 10(3): 208-213, which is incorporated by reference herein in its entirety. The most important property of BM-MSCs, which is used in isolation and purification process, is their physical adherence to the plastic cell culture plate. A variety of techniques have been used for isolation and enrichment of MSCs, including antibody-based cell sorting, low and high-density culture techniques, positive negative selection method, frequent medium changes, and an enzymatic digestion approach.

Briefly, human bone marrow may be obtained from patients and collected aseptically. The buffy coat is isolated by centrifugation (450×g, 10 min), suspended in 1.5 mL PBS, and used for culture. The separated buffy coat is layered onto an equal volume of Ficoll (GE health care, USA) and centrifuged (400×g, 20 min). Cells at the interface are removed, and washed twice in sterile PBS. Human bone marrow progenitor cells are cultured on tissue treated culture plates in DMEM medium supplemented with 10% FBS and penicillin/streptomycin (50 U/mL and 50 mg/mL, Gibco-Invitrogen, Carlsbad, USA; respectively). The plates are maintained at 37° C. in a humidified atmosphere containing 5% CO₂ for 48 h. To exchange the medium, the plates are washed with PBS in order to remove non-adhered cells and the medium is replaced. The cultures are maintained for an additional week with one medium exchange. To characterize the adherent cells, osteoblastic differentiation is induced by culturing confluent human MSCs for 3 weeks in osteoblastic differentiation media (all from Sigma) and after three weeks, the cells are stained by Alizarin. To induce adipocyte differentiation, confluent MSCs are cultured 1 to 3 weeks in differentiation medium, and lipid droplet staining is carried out by S Red Oil (Sigma). Flow cytometry is used to assess the immune profile of MSCs, using the standard for MSC as described by the International Society for Cellular Therapy (ISCT). Cells (P2-3) are harvested, pelleted and resuspended in 1% bovine serum albumin (BSA in PBS), and counted. Cells are stained directly with PE (phycoerythrin) and conjugated antibodies against CD14, CD34, CD45, CD90, CD105 and CD73 (ebioscience, Germany). An appropriate isotype-matched control antibody named mouse IgG1 K Iso control (ebioscience, Germany) is used in all analyses. Cells are analyzed on FACS flow cytometry using Cell Quest Software (Becton Dickinson, UK). See Baghaei et al., 2017, cited above.

Another convenient source for human MSCs is the umbilical cord, which is discarded after birth and provides an easily accessible and non-controversial source of stem cells for therapy (El Omar et al., 2014, Tissue Eng Part B Rev 20(5): 523-544). Human umbilical cord MSCs (hUC-MSCs) have been validated for safety and efficacy in human clinical trials as suspensions (Bartolucci et al., 2017, Circ Res, 121(10), 1192-1204). Moreover, hUC-MSCs have been successfully used in experimental animal disease models (Zhang et al., 2017, Cytotherapy 19(2): 194-199).

Methods for isolating MSCs from umbilical cords are known in the art and are described, for example, in U.S. Pat. No. 9,903,176, which is incorporated by reference herein in its entirety. The human umbilical cord comprises the umbilical artery, the umbilical veins, Wharton's Jelly, and the subepithelial layer. In some embodiments, the hUC-MSCs are isolated from the subepithelial layer of the human umbilical cord. In some embodiments, the hUC-MSCs are isolated from Wharton's Jelly of the human umbilical cord. Various cellular markers may be used to identify hUC-MSCs isolated from the subepithelial layer. For example, in some embodiments, the hUC-MSCs isolated from the subepithelial layer express one or more cell markers selected from CD29, CD73, CD90, CD146, CD166, SSEA4, CD9, CD44, CD146, and CD105. In a particular embodiment, the hUC-MSCs express CD73. In some embodiments, the hUC-MSCs isolated from the subepithelial layer do not express one or more cell markers selected from CD45, CD34, CD14, CD79, CD106, CD86, CD80, CD19, CD117, Stro-1, HLA-DR, HLA-DP and HLA-DQ. In a particular embodiment, the hUC-MSCs do not express HLA-DR, HLA-DP or HLA-DQ. In some embodiments, the cell sheets described herein are prepared with mesenchymal stem cells (MSCs) with low HLA expression, e.g. less than 5%, 4%, 3%, 2%, 1%, 0.5%, 0.4%, 0.3%, 0.2%, or 0.1% of the MSCs in the cell sheet express HLA (e.g. HLA-DR, HLA-DP and/or HLA-DQ).

II. Chondrogenic Cell Sheets Produced from MSCs

In certain aspects the present disclosure relates to a chondrogenic cell sheet prepared from mesenchymal stem cells (MSCs). The term “chondrogenic cell sheet” as used herein refers to a cell sheet obtained by growing mesenchymal stem cells on a temperature-responsive cell culture support in vitro and treating the cell sheet with one or more growth factors (e.g. transforming growth factor beta (TGFβ) and/or a bone morphogenic protein (BMP)) that induce chondrogenesis to produce a cell sheet comprising chondrogenically differentiated cells. The term “chondrogenically differentiated cells” as used herein refers to cells derived from MSCs that express sox9, collagen type II, aggrecan, and sulfated proteoglycans in the extracellular matrix (ECM).

The chondrogenic cell sheet may comprise at least two layers of confluent chondrogenically differentiated cells, for example, at least 2, 3, 4, 5, 6, 7, 8, 9 or 10 layers of confluent chondrogenically differentiated cells. In some embodiments, the chondrogenically differentiated cells on the basal side of the cell sheet express laminin. The term “basal side” as used herein refers to the side of the cell sheet that is in contact with the solid cell culture support during culture of the cell sheet. The basal side of the cell sheet is placed in contact with cartilage tissue of a host during transplantation of the cell sheet to the host. Laminins are high-molecular weight heterotrimeric proteins that are secreted into the extracellular matrix. These heterotrimeric proteins intersect to form a cross-like structure that can bind to other cell membrane and extracellular matrix molecules, including other laminins. Accordingly, laminins play an important role in cell adhesion.

In some embodiments, the chondrogenic cell sheet exhibits physical adhesion to a cartilage surface after transplantation to the cartilage surface. For example, in some embodiments, the chondrogenic cell sheet has adhered to a cartilage surface 30 minutes, 1 hour, 2 hours, 3 hours, 6 hours, 12 hours, or 1, 2, 3, 4, 5, 6, 7, 8, 9 or 10 days after the chondrogenic cell sheet has been transplanted to the cartilage surface.

In some embodiments, the chondrogenic cell sheet has a hyaline-like cartilage phenotype. Characteristics of a hyaline-like cartilage phenotype include, but are not limited to an extracellular matrix (ECM) comprising type II collagen and proteoglycans (which enable resistance to shear, compressive, and tensile forces), expression of SOX9, aggrecan and COL2A1, low expression of type I collagen, a high COL2A1/COL1A1 ratio, nuclei in lacunae structures, rounded cell structure and low cellular densities relative to the ECM.

In some embodiments, the chondrogenically differentiated cells in the chondrogenic cell sheet are aggregated or physically contiguous. In some embodiments, the MSCs used to prepare the chondrogenic cell sheets are human bone marrow MSCs. The chondrogenic cell sheets described herein may be prepared by culturing MSCs on a temperature-responsive culture dish (TRCD), harvesting the MSCs as an intact sheet by temperature shift without any enzyme treatment, allowing the cell sheets to contract, and then treating the cell sheet with one or more growth factors (e.g. TGFβ and/or a BMP) to induce chondrogenesis. The MSC sheets maintain their integrity and shape by retaining tissue-like structures, actin filaments, extracellular matrix, intercellular proteins, and high cell viability, all of which are related to improved cell survival and cellular function. Accordingly, the chondrogenic cell sheets described herein may comprise structural features that improve cell survival and cell function, including native extracellular matrix, cell adhesion proteins and cell junction proteins. Thus, the chondrogenic cell sheets prepared by the methods described herein have several beneficial characteristics compared to cell sheets produced by other methods. For example, the chemical disruption method is unable to maintain tissue-like structures of cells as well as cell-cell communication, since enzyme treatment disrupts the extracellular and intracellular proteins (cell-cell and cell-ECM junctions). Accordingly, protein cleavage by enzymes reduces cell viability and cellular functions. Physical disruption (i.e., by rubber policeman or media aspiration) produces disruption of cell-cell junctions and disintegration of the cultured adherent sheet into cell aggregates.

In some embodiments, the extracellular matrix comprises one or more proteins selected from the group consisting of fibronectin, laminin and collagen. In some embodiments, the cell junction proteins are selected from the group consisting of Integrin-β1, Connexin 43, β-catenin, and N-cadherin.

In some embodiments, the chondrogenic cell sheet consists of chondrogenically differentiated cells. In some embodiments, the chondrogenic cell sheet consists essentially of chondrogenically differentiated cells. In some embodiments, at least 50%, 60%, 70%, 80%, 90%, 95%, 96%, 97%, 98% or 99% of cells in the chondrogenic cell sheet are chondrogenically differentiated cells. In some embodiments, 100% of the cells in the chondrogenic cell sheet are chondrogenically differentiated cells.

The MSCs used to prepare the chondrogenic cell sheets may be added to the culture solution on the temperature-responsive polymer in the cell culture support at various cell densities to optimize formation of the cell sheet or its characteristics. For example, cytokine expression levels in the MSC may be optimized by controlling the initial cell density of the MSCs in the cell culture support (e.g. TRCD). In some embodiments, increasing the initial cell density of the MSCs in the cell culture support increases cytokine expression (e.g. HGF). In some embodiments, increasing the initial cell density of the MSCs in the cell culture support decreases cytokine expression. In some embodiments the initial cell density of the MSCs in the cell culture support used for preparation of the cell sheet is from 1×10³/cm² to 9×10⁵/cm². In some embodiments, the initial cell density of the MSCs in the cell culture support is at least 1×10³, 5×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, or 9×10⁵ cells/cm². Any of these values may be used to define a range for the initial cell density of the MSCs in the cell culture support. For example, in some embodiments, the initial cell density in the cell culture support is from 1×10³ to 5×10³ cells/cm², 2×10⁴ to 1×10⁵ cells/cm², 4×10⁴ to 1×10⁵ cells/cm², or 1×10³ to 5×10⁴ cells/cm².

The chondrogenic cell sheets described herein may also continue to express extracellular matrix proteins and cell junction proteins after transplantation to a target tissue in a host organism. For example, in some embodiments the cell sheet expresses extracellular matrix proteins and/or cell junction proteins for at least 1, 2, 3, 4, 5, 6, 7, 8, 9, 10, 15, 20, 25 or 30 days after transplantation to a tissue in a host organism. In some embodiments, the cell sheet expresses the extracellular matrix proteins and/or cell junction proteins for at least 1, 2, 3, 4, 5 or 6 months after transplantation to a tissue of a host organism. In some embodiments the extracellular matrix proteins expressed in the cell sheet after transplantation are selected from fibronectin, laminin and collagen. In some embodiments the cell junction proteins expressed in the cell sheet after transplantation are selected from Vinculin, Integrin-β1, Connexin 43, β-catenin, Integrin-linked kinase and N-cadherin.

Current stem cell therapies often use cultured stem cells isolated from biopsies as injectable cell suspensions (Bayoussef et al., 2012, J Tissue Eng Regen Med, 6(10)). Injected cell suspensions typically exhibit lower engraftment into and retention within diseased organs or tissues (Devine et al., 2003, Blood, 101(8), 2999-3001). Loss of intact ECM and cell-cell junctions (i.e., communication) in stem cell suspensions through enzymatic disruption at harvest compromises stem cell function, engraftment and survival in vivo, and can limit therapeutic efficacy in vivo. In contrast, the methods of preparing chondrogenic cell sheets described herein preserve intrinsic cell functional structures, improving attachment of the cell sheet to the target tissue after transplantation. For example, in some embodiments, the cell sheet remains attached to the target tissue in the host organism for at least 1, 2, 3, 4, 5, 6, 7, 8, 9, 10, 15, 20, 25 or 30 days after transplantation to a tissue in a host organism. In some embodiments, the cell sheet remains attached to the target tissue in the host organism for at least 1, 2, 3, 4, 5 or 6 months after transplantation to a tissue of a host organism.

The chondrogenic cell sheets described herein also differ from suspension cultures in several ways. Suspension cultures comprise single cells lacking an ECM or cell-cell junctions because these cell adhesive proteins in these cell-cell junctions must be removed (e.g. by proteolytic trypsin treatment) to harvest and suspend cells from culture surfaces commonly used for preparation of the cell suspension culture. In contrast to singe cell suspensions, the chondrogenic cell sheets described herein contain both an endogenous cell-produced ECM and intact cell-cell junctions among the cells that are generated during formation of the cell sheet. The endogenous ECM and intrinsic cell-cell junctions retained during cell sheet formation, fabrication and handling facilitate retention of important properties for their phenotypic preservation, cell functions and adhesion of the chondrogenic cell sheet to target tissue (e.g. articular cartilage) during transplantation to a host organism.

III. Methods for Producing Chondrogenic Cell Sheets In Vitro

In certain aspects, the disclosure relates to a method of preparing a chondrogenic cell sheet comprising: a) culturing mesenchymal stem cells (MSCs) on temperature responsive cultureware until confluent to form a cell sheet; b) detaching the cell sheet by temperature reduction and allowing the cell sheet to contract, forming a contracted cell sheet; c) contacting the contracted cell sheet with a culture surface; and d) treating the contracted cell sheet on the culture surface with chondrogenic medium and culturing to form a chondrogenic cell sheet.

In some embodiments, the contracted cell sheet is grown on the culture surface for at least 1, 2, 3 or 4 weeks. In some embodiments, the chondrogenic medium comprises a protein selected from the group consisting of transforming growth factor beta (TGFβ) and a bone morphogenic protein (BMP). In some embodiments, the chondrogenic medium comprises one or more of the following media components: Insulin-Transferrin-Selenium (ITS-G), dexamethasone, bovine serum albumin (BSA), L-ascorbic acid 2-phosphate, L-proline, and linoleic acid.

In some embodiments, the MSCs are human bone marrow mesenchymal stem cells (hBM-MSCs). Methods for isolating hUC-MSCs are known in the art and are described herein.

General methods for preparing cell sheets are known in the art and are described, for example, in U.S. Pat. Nos. 8,642,338; 8,889,417; 9,981,064; and 9,114,192, each of which is incorporated by reference herein in its entirety.

The temperature-responsive polymer used to coat the substrate of the cell culture support has an upper or lower critical solution temperature in aqueous solution which is generally in the range of 0° C. to 80° C., for example, 10° C. to 50° C., 0° C. to 50° C., or 20° C. to 45° C.

The temperature-responsive polymer may be a homopolymer or a copolymer. Exemplary polymers are described, for example, in Japanese Patent Laid-Open No. 211865/1990. Specifically, they may be obtained by homo- or co-polymerization of monomers such as, for example, (meth)acrylamide compounds ((meth)acrylamide refers to both acrylamide and methacrylamide), N-(or N,N-di)alkyl-substituted (meth)acrylamide derivatives, and vinyl ether derivatives. In the case of copolymers, any two or more monomers, such as the monomers described above, may be employed. Further, those monomers may be copolymerized with other monomers, one polymer may be grafted to another, two polymers may be copolymerized, or a mixture of polymer and copolymer may be employed. If desired, polymers may be crosslinked to an extent that will not impair their inherent properties.

The substrate which is coated with the polymer may be of any types including those which are commonly used in cell culture, such as glass, modified glass, silicon oxide, polystyrene, poly(methyl methacrylate), polyester, polycarbonate, and ceramics.

Methods of coating the support with the temperature-responsive polymer are known in the art and are described, for example, in Japanese Patent Laid-Open No. 211865/1990. Specifically, such coating can be achieved by subjecting the substrate and the above-mentioned monomer or polymer to, for example, electron beam (EB) exposure, irradiation with γ-rays, irradiation with UV rays, plasma treatment, corona treatment, or organic polymerization reaction.

The coverage of the temperature responsive polymer may be in the range of 0.4-3.0 m/cm², for example, 0.7-2.8 m/cm², or 0.9-2.5 μg/cm². The morphology of the cell culture support may be, for example, a dish, a multi-plate, a flask or a cell insert.

The cultured cells may be detached and recovered from the cell culture support by adjusting the temperature of the support material to the temperature at which the polymer on the support substrate hydrates, whereupon the cells can be detached. Smooth detachment can be realized by applying a water stream to the gap between the cell sheet and the support. Detachment of the cell sheet may be affected within the culture solution in which the cells have been cultivated or in other isotonic fluids, whichever is suitable.

In a particular embodiment, the temperature-responsive polymer is poly(N-isopropyl acrylamide) Poly(N-isopropyl acrylamide) has a lower critical solution temperature in water of 31° C. If it is in a free state, it undergoes dehydration in water at temperatures above 31° C. and the polymer chains aggregate to cause turbidity. Conversely, at temperatures of 31° C. and below, the polymer chains hydrate to become dissolved in water, thereby causing release of the cell sheet from the polymer. In a particular embodiment, this polymer covers the surface of a substrate such as a Petri dish and is immobilized on it. Therefore, at temperatures above 31° C., the polymer on the substrate surface also dehydrates but since the polymer chains cover the substrate surface and are immobilized on it, the substrate surface becomes hydrophobic. Conversely, at temperatures of 31° C. and below, the polymer on the substrate surface hydrates but since the polymer chains cover the substrate surface and are immobilized on it, the substrate surface becomes hydrophilic. The hydrophobic surface is an appropriate surface for the adhesion and growth of cells, whereas the hydrophilic surface inhibits the adhesion of cells and the cells are detached simply by cooling the culture solution.

Culture solutions for mesenchymal stem cells are known in the art and are described, for example, in U.S. Pat. Nos. 9,803,176 and 9,782,439, each of which is incorporated by reference herein in its entirety. In some embodiments, the cell culture solution comprises human platelet lysate (hPL). In some embodiments, the culture solution comprises fetal bovine serum (FBS). In some embodiments, the culture solution comprises ascorbic acid. In some embodiments, the culture solution is a xeno-free medium, i.e. a medium that may contain products obtained from humans but does not contain products obtained from non-human animals. In some embodiments, the culture solution contains at least one product obtained from a non-human animal (e.g. FBS). In some embodiments, the culture solution does not contain a product obtained from a human. In a particular embodiment, the culture solution comprises one or more of Dulbecco's Modified Eagle's Medium (DMEM) (Life Technologies, CA, USA), human platelet lysate (hPL, iBiologics, Phoenix, USA), Glutamax (Life Technologies), MEM Non-Essential Amino Acids Solution (NEAA) (Life Technologies) and an antibiotic, e.g. penicillin streptomycin.

The MSCs (e.g. hBM-MSCs) may be passed through one or more subcultures (i.e. passages) prior to culturing the cells in culture solution on a temperature-responsive polymer which has been coated onto a substrate surface of a cell culture support. In some embodiments, the MSCs (e.g. hBM-MSCs) are passed through 1, 2, 3, 4, 5, 6, 7, 8, 9, 10, 11, 12, 13, 14 or 15 subcultures prior to culturing the cells in culture solution on a temperature-responsive polymer which has been coated onto a substrate surface of a cell culture support. Any of these values may be used to define a range for the number of subcultures. For example, in some embodiments, the MSCs (e.g. hBM-MSCs) are passed through 2 to 10, 4 to 8, or 1 to 12 subcultures prior to culturing the cells on a temperature-responsive polymer. In some embodiments the number of subcultures is less than 1, 2, 3, 4, 5, 6, 7, 8, 9, 10, 11, 12, 13, 14 or 15. In some embodiments, the number of subcultures is at least 1, 2, 3, 4, 5, 6, 7, 8, 9, 10, 11, 12, 13, 14 or 15.

The chondrogenic cell sheet may be prepared in a range of different sizes depending on the application. In some embodiments, the chondrogenic cell sheet has a diameter of at least 1, 2, 3, 4, 5, 6, 7, 8, 9, 10, 15 or 20 cm. Any of these values may be used to define a range for the size of the chondrogenic cell sheet. For example, in some embodiments, the chondrogenic cell sheet has a diameter from 1 to 20 cm, from 1 to 10 cm or from 2 to 10 cm. In some embodiments, the chondrogenic cell sheet has an area of at least 1, 2, 3, 4, 5, 6, 7, 8, 9, 10, 15, 20, 30, 40, 50, 60, 70, 80, 90, 100, 150, 200, 250 or 300 cm². Any of these values may be used to define a range for the size of the chondrogenic cell sheet. For example, in some embodiments, the chondrogenic cell sheet has an area from 1 to 100 cm², 3 to 70 cm², or 1 to 300 cm². The methods described herein result in a chondrogenic cell sheet in which the surface area of the chondrogenic cell sheet is much greater than its thickness. For example, in some embodiments the ratio of the surface area of the chondrogenic cell sheet to its thickness is at least 10:1, 100:1, 1000:1, or 10,000:1. The chondrogenic cell sheets described herein comprise one or more layers of confluent chondrogenically differentiated cells, for example, 1, 2, 3, 4, 5, 6, 7, 8, 9 or 10 layers of chondrogenically differentiated cells. In some embodiments, the chondrogenic cell sheet comprises fewer than 2, 3, 4, 5, 6, 7, 8, 9 or 10 layers of chondrogenically differentiated cells. In some embodiments, the chondrogenic cell sheet comprises at least 1, 2, 3, 4, 5, 6, 7, 8, 9 or 10 layers of chondrogenically differentiated cells.

IV. Methods of Using Chondrogenic Cell Sheets

In some aspects, the present disclosure relates to a method of transplanting a chondrogenic cell sheet to a subject in need thereof, comprising applying a chondrogenic cell sheet as described herein to a tissue of a subject.

In some aspects, the present disclosure relates to a method of repairing cartilage tissue in a subject in need thereof, comprising applying a chondrogenic cell sheet as described herein to cartilage of the subject, thereby repairing the cartilage tissue in the subject. In some embodiments of the methods described herein, the cartilage is articular cartilage. In some embodiments, the subject has a focal cartilage defect, e.g. a focal articular cartilage defect. The term “focal cartilage defect” as used herein refers to damage to cartilage that is limited to a well-defined area. In some embodiments, the subject has a symptomatic cartilage defect, e.g. a symptomatic articular cartilage defect. In some embodiments, the symptomatic cartilage defect is caused by acute or repetitive trauma. In some embodiments, the subject has a degenerative joint disease, (e.g. osteoarthritis) or is at risk of developing a degenerative joint disease (e.g. osteoarthritis).

In some aspects, the present disclosure relates to a method of treating or preventing a joint disease in a subject in need thereof, comprising applying a chondrogenic cell sheet as described herein to a joint in the subject, thereby treating or preventing the joint disease in the subject. As used herein, the terms “treat,” “treating,” and “treatment” are all intended to refer to an amelioration or reversal of at least one measurable physical parameter related to a joint disease. The terms “treat,” “treating,” and “treatment,” can also refer to preventing the progression, or at least slowing down the progression of the joint disease. In one embodiment, “treat,” “treating,” and “treatment” refer to a reduction or complete alleviation of pain associated with the joint disease, disorder, or condition. In another embodiment, “treat,” “treating,” and “treatment” refer to a reduction of joint inflammation. In yet another embodiment, “treat,” “treating,” and “treatment” refer to inhibiting or reducing the degradation of cartilage in a joint, such as articular cartilage in a synovial joint. And in yet another embodiment, “treat,” “treating,” and “treatment” refer to an alleviation of one or more symptoms associated with the joint disease, such as joint pain, joint swelling, joint stiffness, inflammation, difficulty in joint movement, and reduced range of motion. In some embodiments, the subject has the joint disease as the time that a chondrogenic cell sheet as described herein is first applied to the subject. In some embodiments, the chondrogenic cell sheet is applied to the subject before the joint disease (e.g. osteoarthritis) occurs, for example to prevent occurrence of the joint disease.

In some embodiments, the joint is selected from a synovial joint and a cartilaginous joint. The term “synovial joint” refers to the most common and movable type of joint in the body of a mammal. Synovial joints include hinge joints (e.g., elbow and knee), pivot joints (e.g., atlas and axis bones at the top of the neck), ball and socket joints (e.g., hip), saddle joints (e.g., carpometacarpal joint of the thumb), condyloid joints (e.g., wrist, metacarpophalangeal joints, metatarsophalangeal joint), and gliding joints (e.g., intercarpal joints in the wrist). Non-limiting examples of synovial joints include, but are not limited to, knee, wrist, shoulder, hip, elbow, facet, carpal-metacarpal, and tarsal/metatarsal joints. “Cartilaginous joints” are joints connected entirely by cartilage, such as the manubrio-sternal joint (sternum) and amphiarthoses joints, such as intervertebral discs. In certain embodiments, the joint disease affects a synovial joint. In other embodiments, the joint disease affects a cartilaginous joint.

In some embodiments the joint disease is a degenerative joint disease, e.g. osteoarthritis. As used herein, “osteoarthritis” refers to a form of arthritis occurring in synovial joints. It is usually a chronic condition, and occurs when the protective cartilage, known as articular cartilage, on the ends of bones that come together to form joints wears down and/or is degraded. “Articular cartilage” refers to the tissue at the ends of bones in joints, which provides frictionless contact between the bones in a joint during movement. Articular cartilage is composed of two major components: collagen and proteoglycans. The breakdown of cartilage in synovial joints can be caused by a number of factors including, but not limited to, proteases, aging, being overweight, and genetic defects in cartilage formation. In some embodiments, the joint disease is selected from the group consisting of joint inflammation, osteoarthritis, rheumatoid arthritis, and chondromalacia patellae.
the subject is human.

In certain aspects the disclosure relates to a method of preventing osteoarthritis in a subject having a symptomatic cartilage defect caused by acute or repetitive trauma, the method comprising applying a chondrogenic cell sheet as described herein to the cartilage having the defect, thereby preventing the osteoarthritis in the subject.

In some embodiments, the tissue to which the chondrogenic cell sheet is applied is cartilage, e.g. articular cartilage. In some embodiments, the tissue to which the chondrogenic cell sheet is applied is bone, e.g. subchondral bone.

One advantage of the chondrogenic cell sheets described herein is that the extracellular matrix of the applied cell sheet acts as a natural adhesive to bind the cell sheet to cartilage tissue of the subject, such that suturing or stitching is not required to adhere the cell sheet to the tissue. A support membrane or other devices may be used to transfer the chondrogenic cell sheet to the cartilage tissue of the subject and then removed after sheet transfer. The supports can be, for example, poly(vinylidene difluoride) (PVDF), cellulose acetate, cellulose esters, plastic and metal. The chondrogenic cell sheets readily adhere to target tissue, self-stabilizing without suturing after being placed directly onto the target tissue for a short period of time. For example, in some embodiments, the chondrogenic cell sheet adheres to the target tissue within 5, 10, 15, 20, 25, or 30 minutes after contact with the tissue. Once the chondrogenic sheet has adhered to the uterine tissue, the support membrane may be removed.

In certain embodiments, the chondrogenically differentiated cells in the cell sheet are allogeneic to the subject, i.e. are isolated from a different individual from the same species as the subject, such that the genes at one or more loci are not identical. In certain reported cases, cell sheets derived from MSCs seemingly avoid allogeneic rejection in humans and in animal models (Jiang et al., 2005, Blood, 105(10), 4120-4126). Thus, the chondrogenic cell sheets described herein may be used in allogeneic cell therapies as an off-the-shelf product. In other embodiments, the chondrogenically differentiated cells in the cell sheet are allogeneic to the subject to which the chondrogenic cell sheet is applied. In some embodiments, the subject is human.

Allogeneic cell sources must be capable of eliciting meaningful therapies under standard immunologic competence in host patient allogeneic tissues. This includes reliable cell homing to and fractional dose engraftment or retention for sufficient duration at the tissue site of therapeutic interest (Leor et al., 2000, Circulation, 102(19 Suppl 3), III 56-61). Current estimates are that when stem cell suspensions are administered to a subject, less than 3% of injected stem cells are retained in damaged myocardium 3 days post-injection following ischemic injury (Devine et al., 2003, Blood, 101(8), 2999-3001). Additionally, most administered cells from a cell suspension that engraft into target tissue will die within the first few weeks (Reinecke & Murry, 2002, J Mol Cell Cardiol, 34(3), 251-253). In contrast, the chondrogenic cell sheets described herein stably engraft at high fractional retention to host tissue 3 days after transplantation. Thus the chondrogenic cell sheets described herein provide distinct advantages over injected or administered mesenchymal stem cell suspensions.

More than one chondrogenic cell sheet may be applied to the cartilage tissue of a subject in the methods described herein. In some embodiments, 1, 2, 3, 4, 5, 6, 7, 8, 9, 10 or more chondrogenic cells sheets may be applied to the cartilage tissue (e.g. articular cartilage tissue) of a subject Any of these values may be used to define a range for the number of chondrogenic cell sheets applied to the cartilage tissue of a subject. For example, in some embodiments, 2-4, 3-5 or 1-10 chondrogenic cell sheets are applied to the cartilage tissue (e.g. articular cartilage tissue) of a subject.

### EXAMPLES

### Example 1

Materials and Methods

Cell Culture

Passage 4 human bone marrow derived mesenchymal stem cells (hBM-MSCs), purchased from Lonza at Passage 2, were plated at 3,000 cells/cm² in growth media containing Dulbecco's Modified Eagle's Medium (DMEM, Life Technologies, CA, USA) supplemented with 10% fetal bovine serum (FBS, Thermo Fisher Scientific, MA, USA), 1% penicillin streptomycin (PS, Life Technologies), and 5 ng/mL basic fibroblast growth factor (bFGF, PeproTech, NJ, USA) and incubated in a humidified environment at 37° C., 5% CO₂. Media was changed at 1 day and 5 days, and cells were cultured until 90% confluent, approximately 7 days. Cells were passaged using 0.05% Trypsin-EDTA (Gibco, NY, USA) and the cell suspensions were counted using a hemocytometer.

Cell Sheet and Pellet Fabrication and Chondrogenic Differentiation

Passage 5 hBM-MSCs were aliquoted in 20% FBS growth media at 2.5×10⁵ cells in 15 mL Eppendorf tubes for pellet culture, seeded into 1 μm pore 6-well cell culture inserts for monolayer culture, and seeded into 35 mm UpCell dishes (Nunc, Thermo Fisher Scientific, Denmark) for cell sheet culture, both at 6.7×10⁴ cells/cm². For pellet fabrication, tubes were spun at 500×g for 10 minutes. Caps were loosened and cells were incubated at 37° C., 5% CO₂ for 3 days to allow for pellet formation. For cell sheet fabrication, cells were cultured for 5 days. At 5 days, cell sheets were left at 20° C. for 1 hour, then detached with forceps. For re-plating cell sheets, 1 μm pore cell culture inserts (Falcon, NE, USA) were conditioned with FBS overnight prior to re-plating the cell sheets. Inserts were washed twice with 1×PBS (Gibco) and kept in a standard incubator. Detached cell sheets were transferred to the conditioned cell culture inserts using OHP film (Apollo, NY, USA) to ensure basal contact with the insert well. Cell sheets were incubated in 20 μL growth media at 37° C., 5% CO₂ for 1 hour. After 1 hour, fresh cell growth media was added to the sheets and they were incubated at 37° C., 5% CO₂ for 3 days for cell attachment. After the 3-day incubation step, chondrogenic samples were induced with chondrogenic medium, control samples were kept in cell growth media, and all samples were transferred to a hypoxia incubator (37° C., 5% CO₂, 5% O₂). Chondrogenic medium contained Dulbecco's Modified Eagle's Medium (DMEM, Life Technologies) supplemented with 10 ng/mL transforming growth factor beta-3 (TG933, Thermo Fisher Scientific), 200 ng/mL bone morphogenic protein-6 (BMP6, PeproTech), 1% Insulin-Transferrin-Selenium (ITS-G, Thermo Fisher Scientific), 1% PS (Life Technologies), 1% non-essential amino acids (NEAA, Thermo Fisher Scientific), 100 nM dexamethasone (MP Biomedicals, OH, USA), 1.25 mg/ml bovine serum albumin (BSA, Sigma-Aldrich, MO, USA), 50 μg/mL L-Ascorbic acid 2-phosphate (Sigma-Aldrich), 40 μg/mL L-proline (Sigma-Aldrich), and 5.35 μg/mL Linoleic acid (Sigma-Aldrich). For chondrogenic and control samples, media was changed twice a week for the duration of differentiation (0 days −4 weeks).

Histological Analysis

After fixation with 4% paraformaldehyde (PFA, Thermo Scientific) for 15 min and paraffin embedded. Embedded samples were sectioned at 4 μm and stained with Alcian blue and Safranin-O. Alcian blue is a polyvalent basic dye used to stain acidic polysaccharides such as glycosaminoglycans in cartilage. Safranin O is a basic dye that stains components of articular cartilage (e.g. proteoglycans, chondrocytes and type II collagen) varying shades of red. To detect early chondrogenesis, samples were stained with Alcian blue (EMD Millipore, MA, USA) for 10 min. To detect mature chondrogenesis, Safranin-O staining was conducted according to standard methods. Briefly, samples were stained for 4 min with Wiegert's Iron Hematoxylin (Sigma-Aldrich), 5 min with 0.5 g/L Fast green (Sigma-Aldrich), and 8 min with 0.1% Safranin-O (Sigma-Aldrich). All samples were dried overnight before being imaged with a BX 41 widefield microscope using AmScope Software. The Safranin-O stained slides were used to calculate cell sheet thicknesses and nuclei densities. For each cell sheet slide, 3 pictures were taken along the length of the cell sheet. Using the measurement tools built into the AmScope software, 5 measurements from the apical to basal edge of the sheet were made per picture, and these measurements were evenly spaced out along the sheet. Nuclei counting was done using the same 3 pictures/sheet. Using the measurement tools built into the AmScope software, a 500 μm length of the cell sheet was marked off in the image. The number of nuclei were counted within the marked section using ImageJ software. For cell sheet diameter and volume calculations, macroscopic images of the sheets were analyzed using ImageJ software. Five diameter measurements were made for four cell sheets per group, and thickness measurements were used from the Safranin-O samples mentioned. All measurements were averaged for each sample group.

Immunohistochemical Analysis

For cross-sectional IHC analysis, samples were fixed on the insert membrane with 4% PFA for 15 min and paraffin embedded. Embedded samples were sectioned at 4 μm and stained for collagen type II to detect mature chondrogenesis, MMP13 to detect hypertrophy, and fibronectin and laminin to detect adhesive molecules. Briefly, antigen retrieval was conducted by incubating with a 1:50 dilution of 50× Low pH Target Retrieval Solution (Dako, Agilent Technologies, CA, USA) for 15 min at 106-110° C. at low pressure in a pressure cooker for Collagen Type II samples and with Proteinase K (Dako, Agilent Technologies) for 6 min at RT for MMP13, fibronectin, and laminin samples. Fibronectin and laminin samples were permeabilized with 0.1% Triton X-100 (Sigma-Aldrich) for 10 min at RT. Non-specific binding was blocked for all samples with 10% Normal Goat Serum (Vector Laboratories, CA, USA) at room temperature for 30 min. Collagen type II, MMP13, fibronectin, and laminin samples were then incubated in a 1:200 dilution of Anti-Collagen Type II primary antibody (Thermo Fisher Scientific), a 1:200 dilution of Anti-MMP13 primary antibody (Abcam, Cambridge, UK), a 1:100 dilution of Anti-fibronectin primary antibody (Abcam), or a 1:100 dilution of Anti-laminin primary antibody (Abcam) at 4° C. overnight, respectively. Samples were washed with 1×PBS and incubated in a 1:200 dilution of Goat Anti-Rabbit 488 secondary antibody (Thermo Fisher Scientific) at room temperature, covered, for 2 h. After 2 hours, samples were washed with 1×PBS and mounted with a DAPI-containing mounting medium (Invitrogen, MA, USA). Samples were visualized with a Zeiss Axio widefield microscope and ZEN software. To determine cytoskeletal arrangement, Phalloidin (F-actin) staining was conducted. Briefly, samples were permeabilized with 0.1% Triton X-100 (Sigma-Aldrich) for 15 min and washed with 1×PBS. Samples were then incubated with a 1:100 dilution of Phalloidin Alexafluor 488 (Life Technologies) at RT, covered, for 30 min. Samples were then washed with 1×PBS and incubated with DAPI solution (2 drops/mL, Life Technologies) at RT for 5 min. Samples were washed with 1×PBS and then prepared for mounting. Samples were imaged with a confocal microscope (Nikon A1—NIS Elements AR Software). Images were analyzed and prepared using ImageJ.

RT-PCR Analysis

RNA from samples was extracted using 1 mL TRIzol/sample (Ambion, Life Technologies, CA, USA) with a pestle motor mixer. Total RNA was isolated with the PureLink RNA Mini Kit (Invitrogen, Thermo Fisher Scientific) according to manufacturer instructions. For cDNA synthesis, all samples were synthesized at the same time. Before synthesizing cDNA, the RNA was quantified with a Nanodrop, and all cDNA samples were prepared from 1 μg of RNA/sample. All samples with a purity (A₂₆₀/A₂₈₀) greater than 1.8 were deemed pure enough to continue. cDNA synthesis was conducted with a High Capacity cDNA Reverse Transcriptase Kit (Applied Biosystems, Thermo Fisher Scientific, MA, USA) as per manufacturer instructions. RT-PCR analysis was conducted with TaqMan Universal PCR Master Mix (Applied Biosystems, Thermo Fisher Scientific) using an Applied Biosystems Step One instrument. Gene expression levels were analyzed for the following genes: 1) glyceraldehyde 3-phosphate dehydrogenase (GAPDH, Hs99999905_m1) as a housekeeping gene, 2) SRY-box 9 (Sox9, Hs01001343_g1), 3) collagen type II alpha 1 chain (Col II, Hs00264051_m1), 4) aggrecan (ACAN, Hs00153936_m1), 5) collagen type X alpha 1 chain (ColX, Hs00166657_m1), 6) alkaline phosphatase (ALPL, Hs01029144_m1), 7) matrix metallopeptidase 13 (MMP13, Hs00942584_m1), 11) β-catenin (Hs00355049_m1), 12) bone morphogenic protein 2 (BMP2, Hs00154192_m1), 13) cartilage oligomeric matrix protein (COMP, Hs00164359_m1). All primers were manufactured by Applied Biosystems. Relative gene expression was calculated by the quantitative comparative CT method. Gene expression was normalized to GAPDH expression levels. For chondrogenic differentiation, expression levels are relative to the monolayer 3-week control group, and the time comparison differentiation expression levels are relative to the time 0 monolayer control group.

Post-Differentiation Manipulation and Re-Attachment

For transplantation potential assessing structural changes, 3-weeks chondrogenically differentiated contracted sheets were cut in half using a scalpel. Half of each sheet was immediately fixed in 4% PFA for 15 min and paraffin embedded. The other half of each sheet was re-plated onto a new FBS-coated 35 mm tissue culture plastic dish. To transfer the cell sheet, it was nudged off of the cell culture insert membrane and manually transferred to the new dish with forceps. After being placed on the secondary surface, sheets were incubated in a small amount of chondrogenic medium for 1 h to attach. After 1 hour, fresh chondrogenic media was added and the cell sheets were moved to the hypoxia incubator. Brightfield images were taken of the cells at the edges of the sheet every day during the 3 day culture period. After 3 days, the cell sheet halves were fixed in 4% PFA for 15 minutes and paraffin embedded.

For transplantation potential to the target tissue site, 3-weeks chondrogenically differentiated contracted sheets were cut in quarters using a scalpel. One quarter of each sheet was immediately fixed in 4% PFA for 15 min and paraffin embedded. The other quarters of each sheet were transplanted onto the apical side of fresh ex vivo human articular cartilage pieces (˜2 cm²). The human articular cartilage samples were harvested from human hip articular cartilage during a procedure. After 3 days of co-culture, the cell sheets on cartilage samples were fixed in 4% PFA for 3 days and paraffin embedded.

Statistical Analysis

All statistical analysis was completed on data sets of n=4. All quantitative values are expressed as a mean and standard deviation. Statistical significance was calculated using a 2-tailed, paired, student T-test. Significance was defined as *p<0.05 and **p<0.01.

Results

Chondrogenic Potential of hBM-MSCs

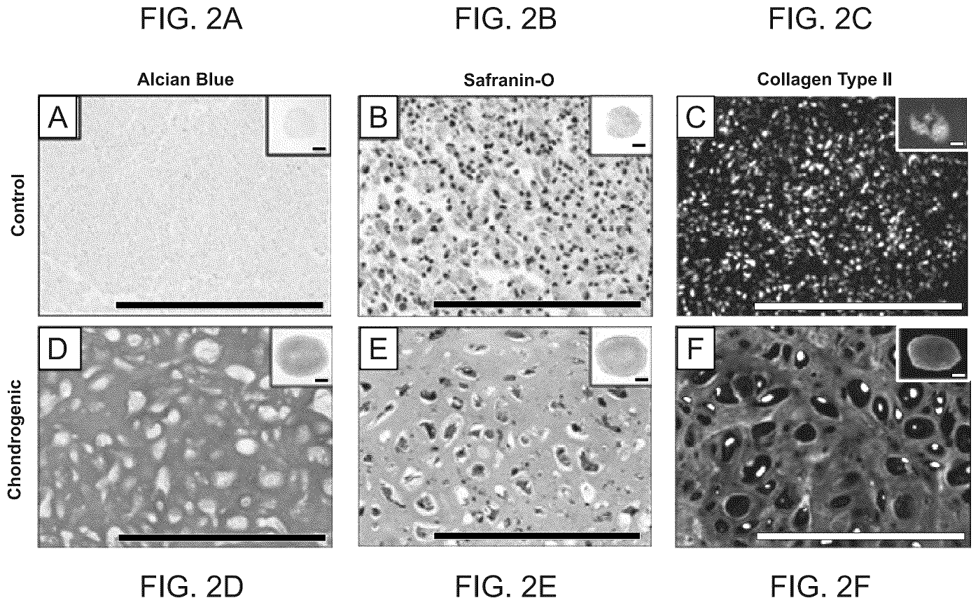

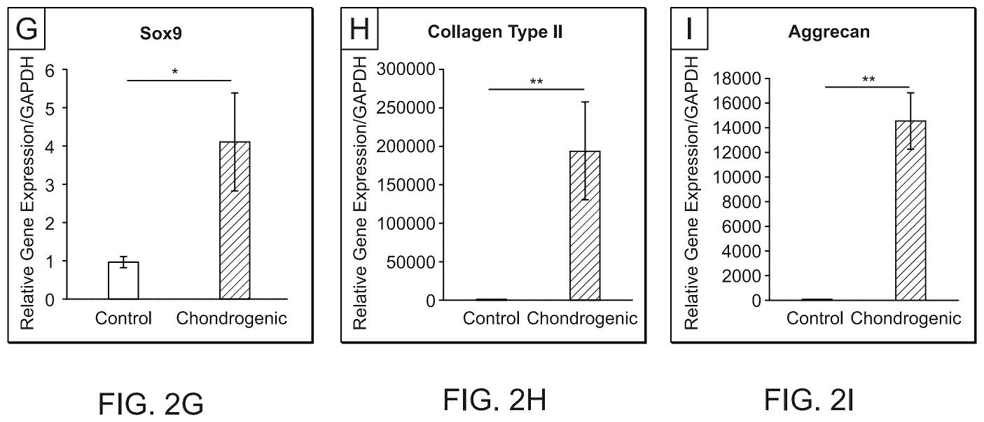

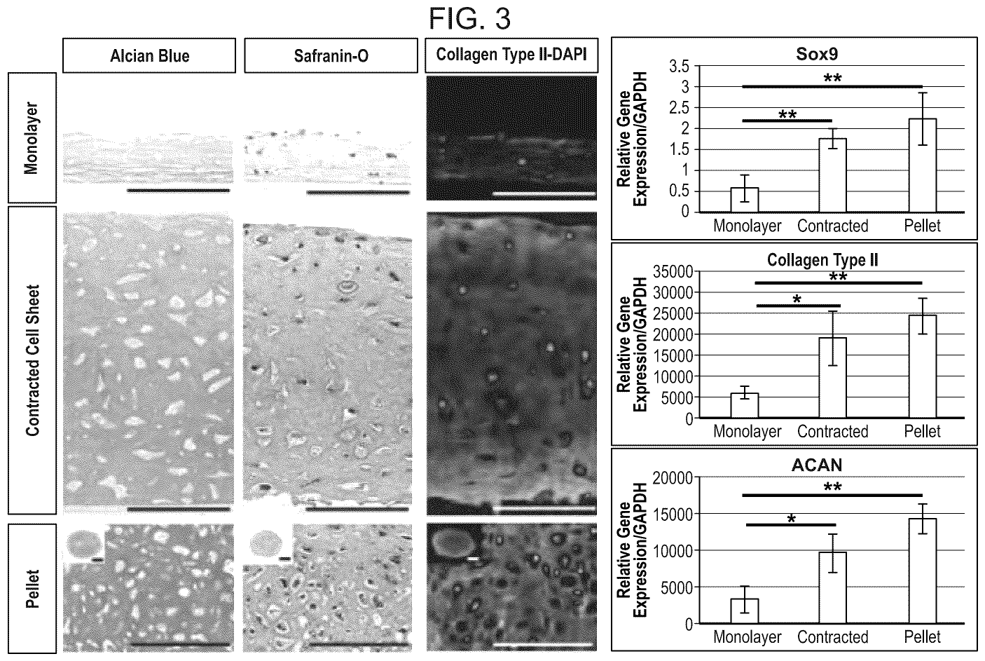

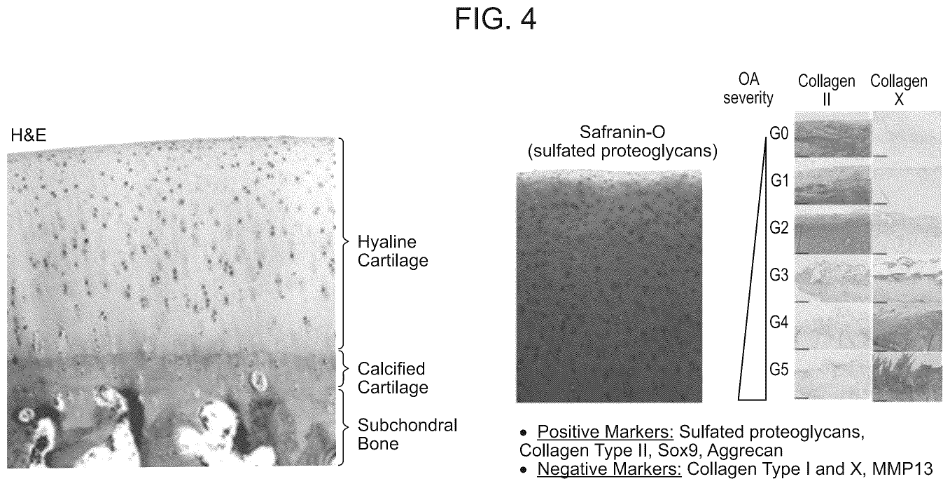

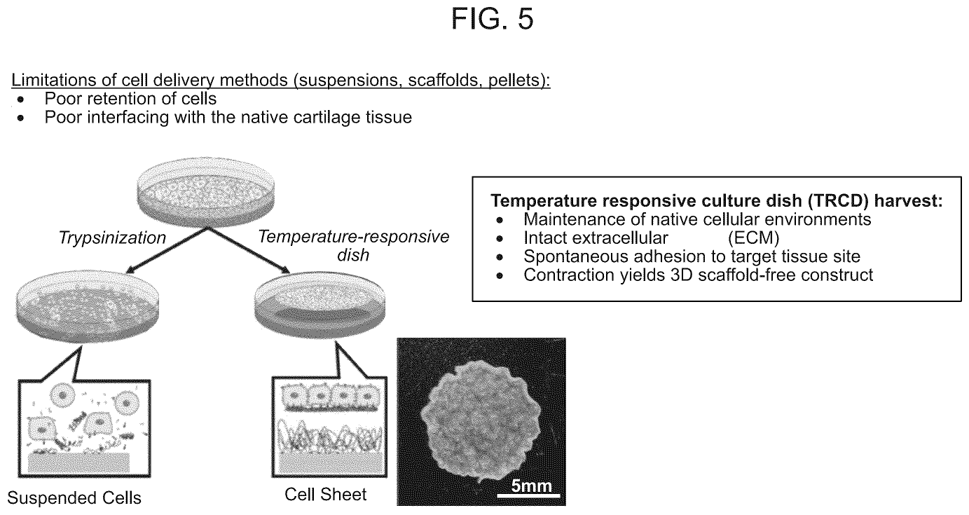

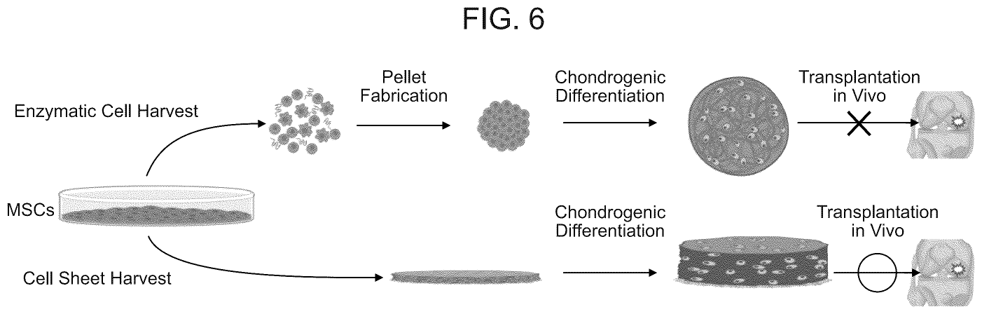

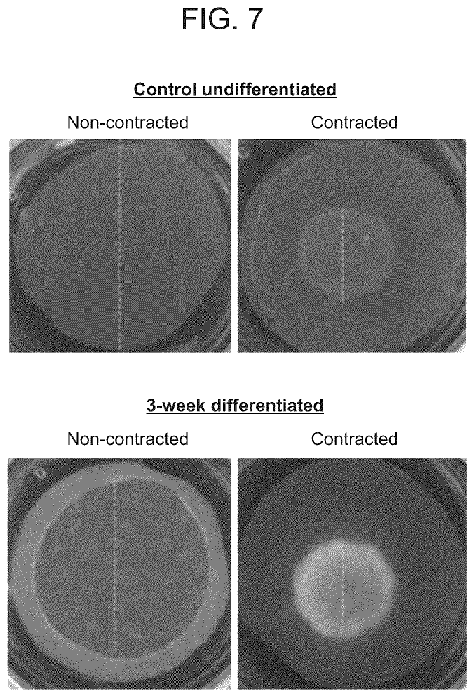

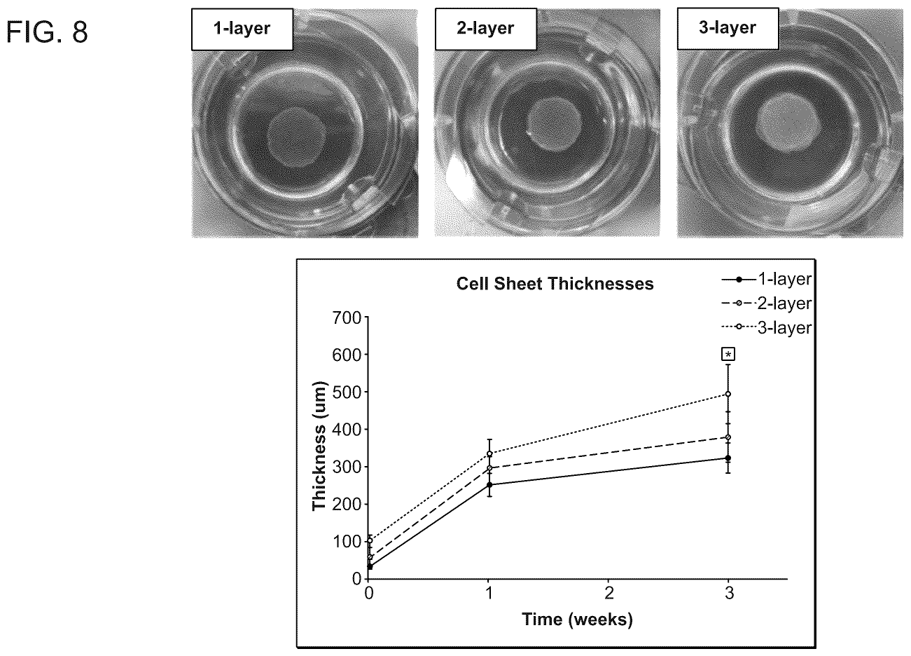

After 3 weeks in chondrogenic media and hypoxia conditions, hBM-MSCs as pellet cultures show expected positive chondrogenic characteristics (FIG. 2). 3-week differentiated pellets are positive for all chondrogenic stains (Alcian blue (FIG. 2D), Safranin-O (FIG. 2E)), Collagen Type II (FIG. 2F)). 3-week control pellets were negative for all chondrogenic stains (FIG. 2A-C). Gene analysis with RT-PCR shows that expression of chondrogenic markers (Sox9 (FIG. 2G)), Collagen Type II (FIG. 2H), Aggrecan (FIG. 2I)) were significantly increased for 3-week differentiated pellets compared to the 3-week control pellets. This data supports the chondrogenic potential of hBM-MSCs.

Endogenous Post-Detachment Contraction of hBM-MSC Cell Sheets

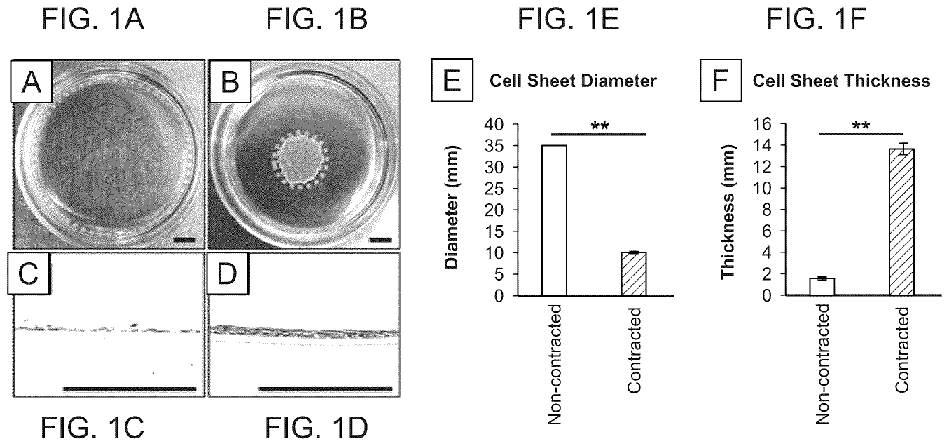

Cell sheets experience spontaneous contraction after detachment from the temperature-responsive culture dishes. Endogenous post-detachment contraction significantly alters the size and structure of the contracted cell sheet (FIG. 1B,D) compared to the non-contracted conditions (FIG. 1A,C). Contracted cell sheets show a 29.2% decrease in diameter (FIG. 1E) and an 883% increase in thickness (FIG. 1F) of the construct compared to non-contracted sheets.

Increased Chondrogenic Potential of hBM-MSCs as Contracted Sheets

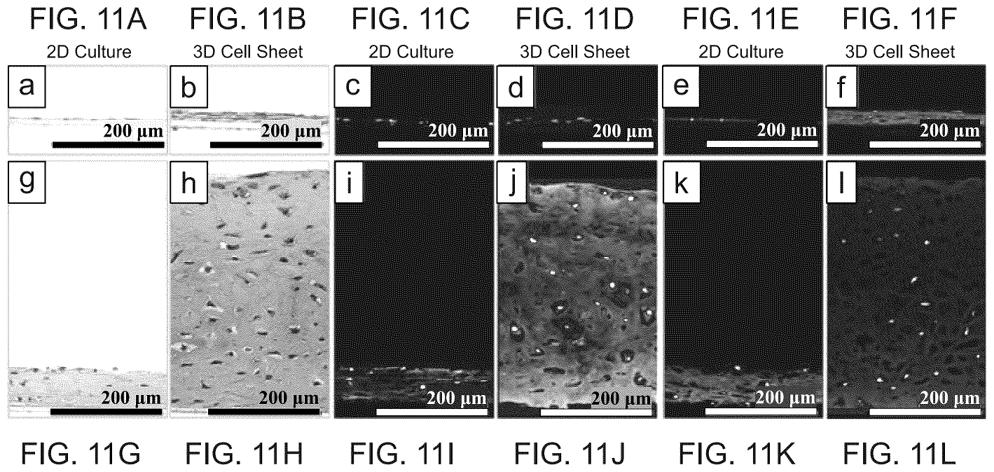

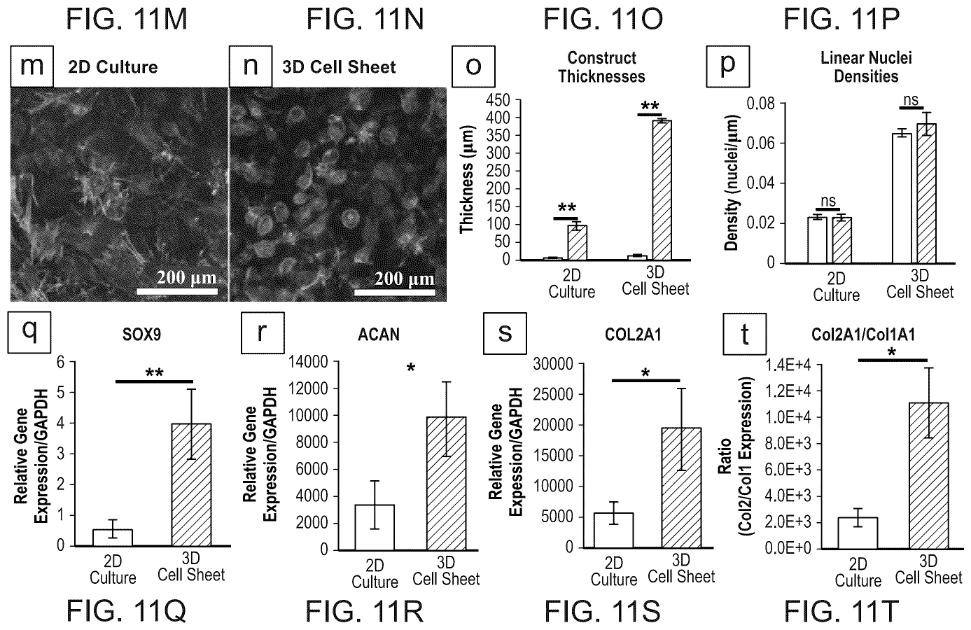

Chondrogenic differentiation for 3 weeks resulted in positive chondrogenesis of cell sheets (FIG. 11). Non-contracted cell sheets showed some slightly positive chondrogenic staining with Alcian blue (FIG. 11), Safranin-O (FIG. 11), and Collagen Type II (FIG. 11) compared with the non-contracted 3-week control sheets (FIG. 11). Positive Alcian blue staining is marked by darker blue color correlated to the acidic mucin content of the sample. Safranin-O stains positive sulphated proteoglycans dark red relative to GAG content and counterstains all other ECM blue. Collagen type II is denoted by red fluorescence (pseudo) and nuclei are counterstained with DAPI (blue). The contracted sheets stained strongly for all chondrogenic markers (Alcian blue (FIG. 11), Safranin-O (FIG. 11), Collagen Type II (FIG. 11)) compared to both the 3-week contracted controls (FIG. 11) and the chondrogenic non-contracted sheets. The differentiated contracted sheets also exhibited lacuna morphology associated with mature hyaline cartilage.

Gene expression for all chondrogenic markers (Sox9 (FIG. 11Q), Col II (FIG. 11S), aggrecan (FIG. 11R)) were significantly increased in contracted sheets compared to non-contracted sheets after 3 weeks of differentiation. Additionally, contracted sheets showed a 30-fold increase in thickness after 3 weeks of differentiation compared to the 23-fold increase in non-contracted sheets (FIG. 11). This increase in thickness after differentiation does not significantly change the number of cells within either the contracted or non-contracted sheet (FIG. 11), which suggests that increased thickness of chondrogenic contracted cell sheet is caused by ECM deposition, which is related to chondrogenesis.

Cytoskeletal Structure Changes for Chondrogenic Differentiation

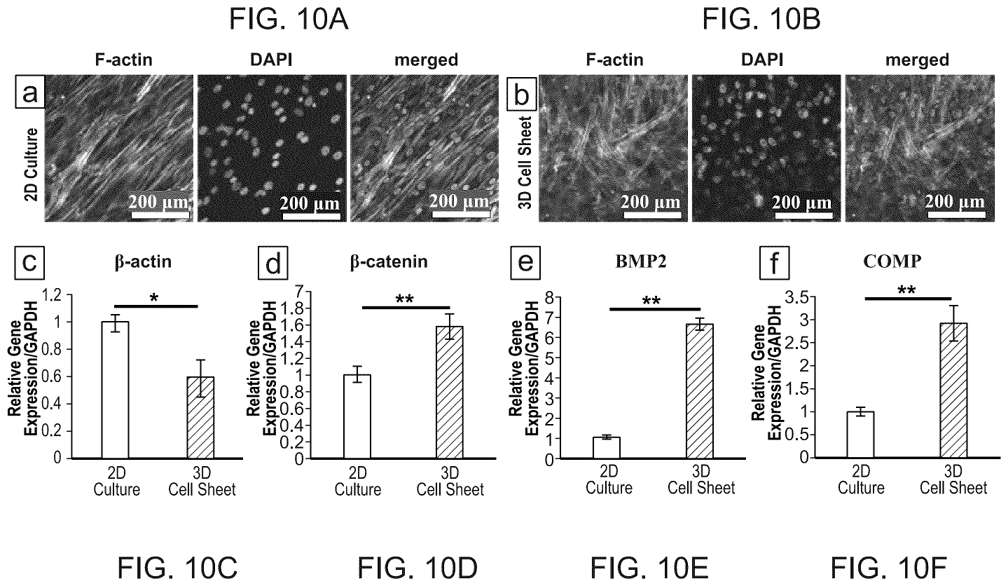

Cytoskeletal arrangement was seen with phalloidin (f-actin) fluorescent staining and nuclei were identified by DAPI (FIGS. 10 and 11). At 0 days, non-contracted sheets show the elongated and aligned cytoskeletal structure associated with standard monolayer culture of MSCs (FIGS. 10 and 11). Conversely, cell sheets that were allowed to contract after detachment exhibited a more random, crossed-fiber structure at 0 days (FIGS. 10 and 11). After 3 weeks of differentiation, the majority of cells in the 3-week differentiated contracted sheet cells exhibit the condensed, rounded cytoskeletal structure associated with mature chondrocytes (FIGS. 10 and 11). Some cells in the non-contracted sheet display this rounded cytoskeletal structure; however, the majority of the cells in the 3-week differentiated non-contracted sheet have a more variable MSC-like structures (FIGS. 10 and 11). In addition to these cytoskeletal changes, contracted sheets showed significant increases in cell-cell interactions (β-catenin (FIGS. 10 and 11)) and ECM components linked to induction and initiation of chondrogenesis (BMP2 (FIGS. 10 and 11), COMP (FIGS. 10 and 11)) compared to non-contracted sheets (FIGS. 10 and 11).

Chondrogenic Differentiation of Cell Sheets Over Time

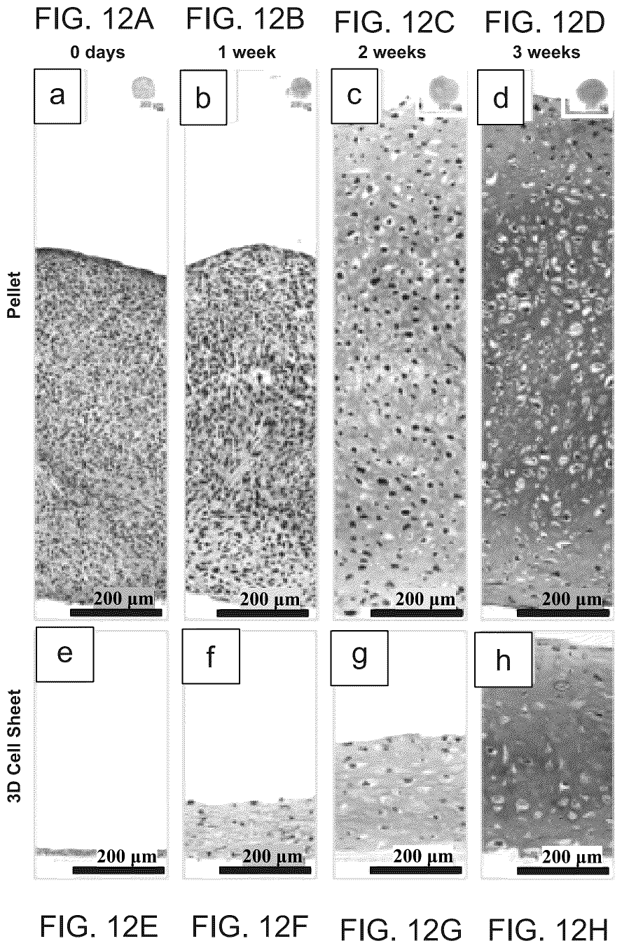

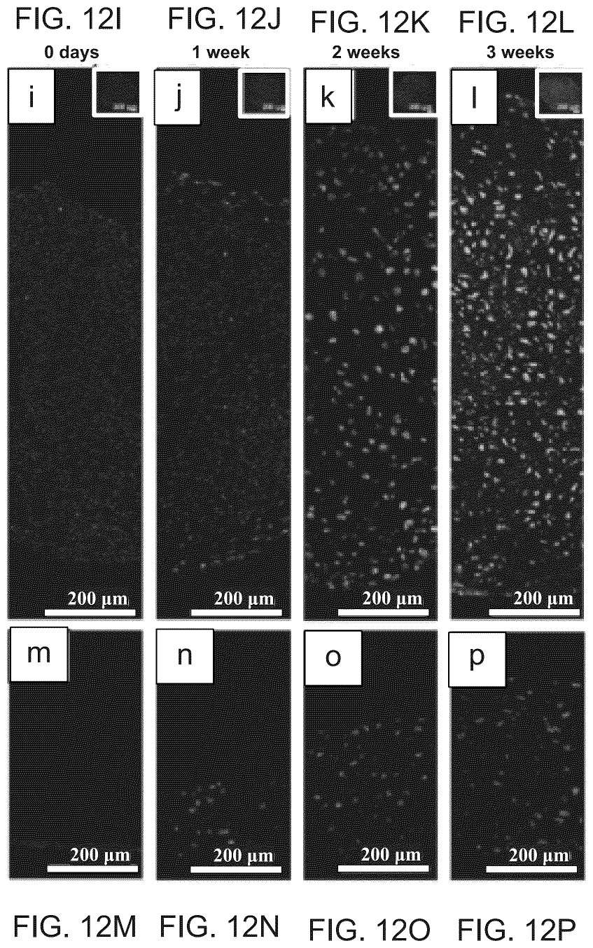

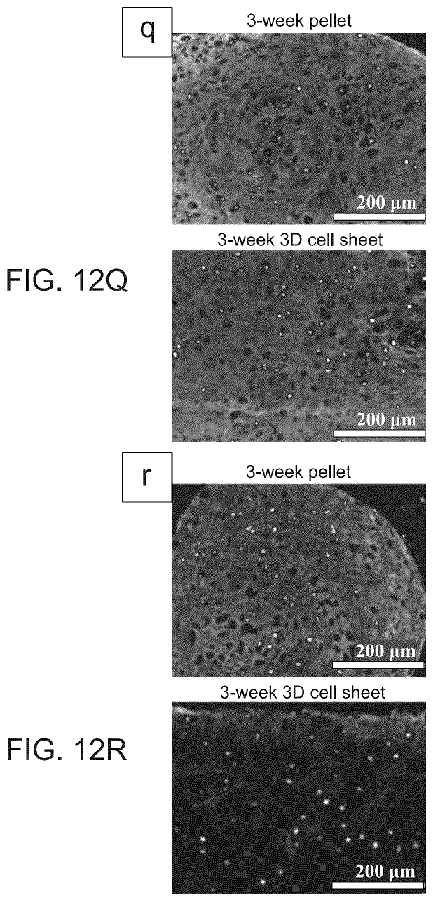

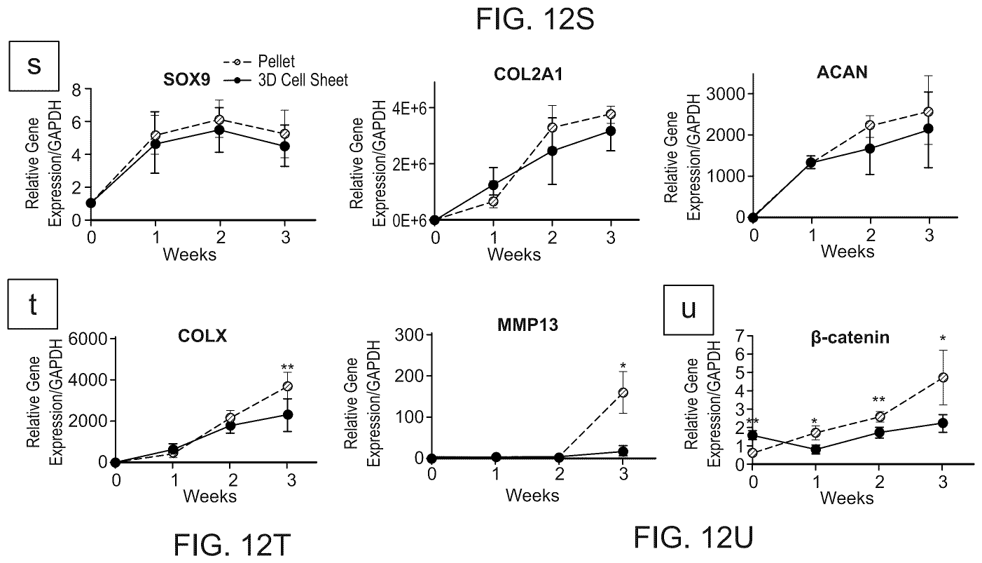

Chondrogenic differentiation of contracted cell sheets and pellets over time show very similar progression of chondrogenesis, with slightly earlier onset of sulphated GAG accumulation seen with contracted cell sheets (FIG. 12). Both pellet cultures and contracted sheets are negative for Safranin-O staining at 0 days (FIG. 12). By 1-week differentiation, the contracted cell sheets (FIG. 12) show greater Safranin-O staining throughout the sample than the pellet cultures (FIG. 12), but staining is very faint for both samples. In conjunction with this Safranin-O staining, contracted sheets showed significant increases in ECM components linked to induction and initiation of chondrogenesis (BMP2 (FIG. 12), COMP (FIG. 12)) compared to pellet cultures. By 3-weeks differentiation, both contracted cell sheets (FIG. 12) and pellets (FIG. 12) stain strongly for Safranin-O and exhibit lacuna morphology. Gene analysis shows a similar trend for chondrogenic markers (Sox9 (FIG. 12) and Collagen Type II (FIG. 12)) expression over 4-week differentiation of contracted sheets and pellets. Sox9 expression for both contracted sheets and pellets increases during early chondrogenesis and then decreases as samples continue differentiating. Chondrogenic marker expression is not significantly different between the contracted sheets and pellets at any time point during differentiation.

Delayed Onset of Hypertrophy of Contracted Cell Sheet

Contracted sheets show a delayed onset of hypertrophic characteristics compared to standard pellet cultures (FIG. 12). Immunohistochemical staining for hypertrophic marker, MMP13, is negative in 0-day pellets and contracted sheets (FIG. 12). MMP13 staining in pellet cultures remains negative or low through 2 weeks (FIG. 12), but is highly expressed in both 3- and 4-week samples (FIG. 12). MMP13 staining is negative or low in contracted sheet samples through 3 weeks (FIG. 12), and is positive only in 4-week samples (FIG. 12). Collagen type X gene expression increases throughout chondrogenic differentiation for both contracted sheets and pellets (FIG. 12). Expression of collagen type X is significantly higher in pellet culture than in contracted sheets at both 3- and 4-week differentiation. In all samples, MMP13 gene expression is low during early chondrogenesis, and begins increasing during later stages of differentiation (FIG. 12). Increases in MMP13 expression occur earlier (between 2 and 3 weeks) in pellet culture than in contracted sheet culture (between 3 and 4 weeks). Expression of MMP13 at 3-week differentiation is significantly higher in pellets compared to contracted cell sheets.

Contracted Cell Sheets after Differentiation can be Manipulated and Transferred to a Secondary Biologic Surface without Losing their Structural Characteristics.

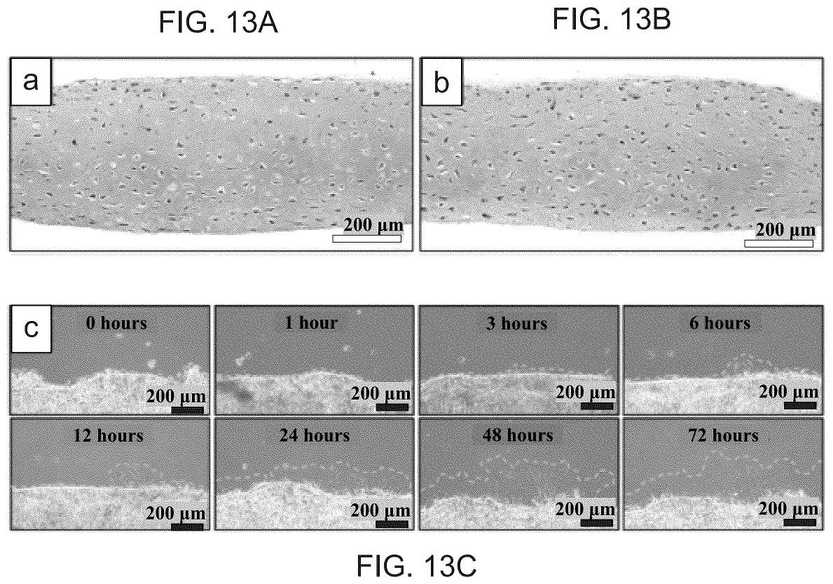

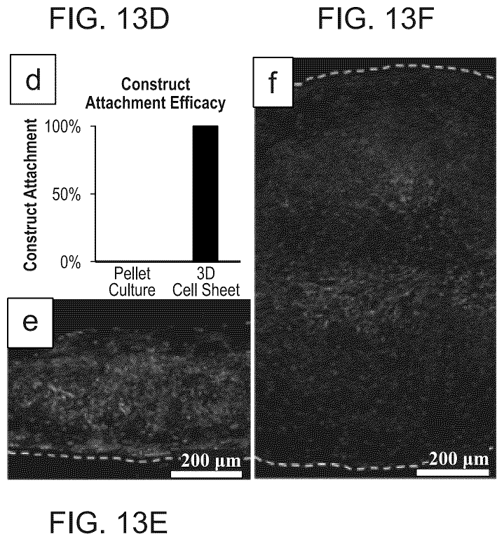

Contracted cell sheets that were differentiated for 3 weeks were able to be manipulated and transferred to a new dish and adhered strongly for 3 days (FIG. 13). After 3 days of secondary culture on the FBS coated dish, cell sheets were harvested, fixed and stained with Safranin-O. This staining showed no discernable effect on the structure and content of the cell sheet after transfer (FIG. 13). Conversely, the pellet cultures transferred to secondary culture dish showed diminished Safranin-O staining and some change in pellet shape (FIG. 13). After 3 days of secondary culture, the contracted sheets maintained localized laminin cell adhesion staining at the basal side of the cell sheet (FIG. 13), where laminin staining of the pellet is localized around the peripheral of the pellet and is weaker at the interface surface compared to cell sheet cultures (FIG. 13). During secondary culture, cell migration can be seen at the edges of the cell sheet as early as 24 hours after transferring the differentiated contracted sheet (FIG. 13), compared to minimal cell migration and delayed progression in pellet cultures (FIG. 13), indicating stronger adhesion to the surface and viability of the cells in contracted sheets than pellet cultures.

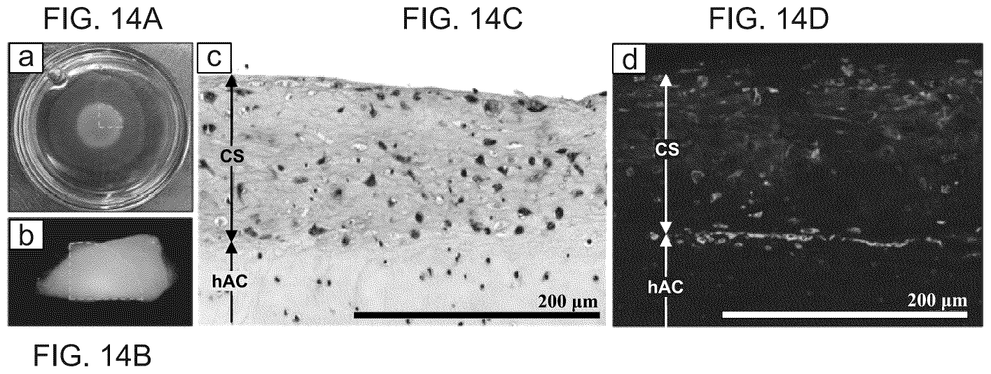

Cell sheets after differentiation strongly adhere to fresh ex vivo cartilage surface for 3 days (FIG. 14). The laminin cell adhesion staining is most intense at the interface between the sheet and the cartilage surface (FIG. 14), indicating strong adhesion and interfacing between the cell sheet and the target tissue.

### Example 2

Many stem cells used for articular cartilage focal defect healing therapies show chondrogenic potential using in vitro pellet culture; however, this potential is rarely preserved when cells are seeded into biomaterial scaffolds. Cell sheet tissue engineering removes scaffold requirements and permits cell transplantation directly from culture. To develop human MSC sheets for cartilage therapies, maintenance of cells' chondrogenic capacity in a sheet form is important.

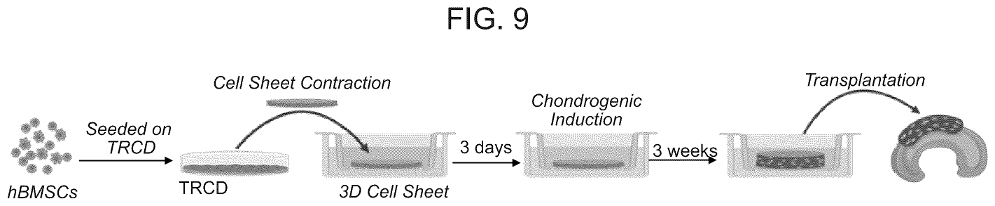

This study aims to fabricate ready-to-use, hyaline-like cartilage constructs from MSCs in vitro using cell sheet technology. This study further demonstrates that cell sheets retain transplantation ability after chondrogenic differentiation to hyaline-like phenotypes, allowing spontaneous adhesion and interfacing with the target tissue site without damaging the structural or chondrogenic characteristics of the construct (FIG. 9). This study describes development of a pre-differentiated hyaline-like cell sheet construct that will be able to reduce the time for transplanted cells to establish hyaline cartilage in vivo for regenerative therapies.

1. Materials and Methods

1.1. Cell Culture

hBM-MSCs, purchased from Lonza at Passage 2, were plated at 3,000 cells/cm² in growth media containing High-Glucose (4.5 g/L) Dulbecco's Modified Eagle's Medium (HG-DMEM) (Life Technologies, CA, USA) supplemented with 10% fetal bovine serum (FBS) (Thermo Fisher Scientific, MA, USA), 1% penicillin streptomycin (PS) (Gibco, NY, USA), and 5 ng/mL basic fibroblast growth factor (bFGF) (PeproTech, NJ, USA) and incubated in a humidified environment (37° C., 5% CO₂). Media was changed at day 1 and day 5, and cells were cultured until 90% confluent, approximately 7 days. Cells were passaged using 0.05% Trypsin-EDTA (Gibco) and the cell suspensions were counted using a hemocytometer. Cells were expanded and banked at Passage 3 and 4 and used for experimentation at Passage 5.

1.2. Cell Sheet and Pellet Fabrication and Chondrogenic Differentiation

Passage 5 hBM-MSCs were aliquoted in 20% FBS growth media at 2.5×10⁵ cells in 15 mL conical tubes for pellet cultures, and seeded at 6.7×10⁴ cells/cm² into 1.0 μm-diameter pore, 6-well cell culture inserts (Falcon, NE, USA) for monolayer cultures and 35 mm diameter UpCell dishes (CellSeed, Tokyo, Japan) for cell sheet cultures. For pellet fabrication, conical tubes were centrifuged at 500×g for 10 minutes. Caps were loosened and cells were transferred to a standard incubator (37° C., 5% CO₂) for 3 days to allow for pellet formation. For cell sheet fabrication, cells were cultured for 5 days. At 5 days, cell sheets were moved to 20° C. for 1 hour, then detached with forceps. For re-plating cell sheets, 1.0 μm-diameter pore, 6-well cell culture inserts were conditioned with FBS overnight prior to re-plating the cell sheets. Inserts were washed twice with 1× phosphate buffered saline (PBS) (Gibco) before sheet transfer. Detached cell sheets were transferred to the conditioned cell culture inserts using overhead projector polyester film (Apollo, NY, USA) to ensure basal contact with insert well culture surfaces and incubated in 20 μL growth media in a standard incubator for 1 hour. After 1 hour, fresh cell growth media was added to the sheets and they were incubated for an additional 3 days to ensure sheet attachment and mirror pellet culture incubation periods. After the 3-day incubation step, chondrogenic samples were induced with chondrogenic medium, control samples were kept in 10% FBS cell growth media, and all samples were transferred to a hypoxia incubator (37° C., 5% CO₂, 5% O₂). Chondrogenic medium contained HG-DMEM supplemented with 10 ng/mL transforming growth factor beta-3 (TGFβ3) (Thermo Fisher Scientific), 200 ng/mL bone morphogenic protein-6 (BMP6) (PeproTech), 1% Insulin-Transferrin-Selenium (ITS-G) (Thermo Fisher Scientific), 1% PS (Life Technologies), 1% non-essential amino acids (NEAA) (Thermo Fisher Scientific), 100 nM dexamethasone (MP Biomedicals, OH, USA), 1.25 mg/mL bovine serum albumin (BSA) (Sigma-Aldrich, MO, USA), 50 μg/mL L-ascorbic acid 2-phosphate (Sigma-Aldrich), 40 μg/mL L-proline (Sigma-Aldrich), and 5.35 μg/mL linoleic acid (Sigma-Aldrich). For chondrogenic and control samples, media was changed twice a week for the duration of differentiation (day 0-3 weeks).

1.3. Histological Analysis

After fixation with 4% paraformaldehyde (PFA) (Thermo Scientific) for 15 min, samples were paraffin embedded. Embedded samples were sectioned at 4 μm. To identify cell morphology, Hematoxylin and Eosin (H&E) staining was conducted according to standard methods. Briefly, samples were stained for 4 min with Mayer's Hematoxylin (Sigma-Aldrich) and 4 min with Eosin (Thermo Scientific). To detect mature chondrogenesis, Safranin-O staining was conducted according to standard methods. Briefly, samples were stained for 4 min with Wiegert's Iron Hematoxylin (Sigma-Aldrich), 5 min with 0.5 g/L Fast green (Sigma-Aldrich), and 8 min with 0.1% Safranin-O (Sigma-Aldrich). All samples were dried overnight before being imaged with a BX 41 widefield microscope (Olympus, Japan) using AmScope Software (USA). Safranin-O stained slide cross sections were used to calculate cell sheet thicknesses and nuclei densities. For each cell sheet slide, 3 pictures were taken along the length of the cell sheet. Using the measurement tools built into the AmScope software, 5 measurements from the apical to basal edge of the sheet were made per picture, and these measurements were evenly spaced out along the sheet. Nuclei counting was done using the same 3 pictures/sheet. Using the measurement tools built into the AmScope software, a 500 μm length of the cell sheet was marked. The number of nuclei were counted within the marked section using ImageJ software (NIH, USA). For cell sheet diameter calculations, macroscopic images of the sheets were analyzed using ImageJ software. Five diameter measurements were made for four cell sheets per group. All measurements were averaged for each sample group.

1.4. Immunohistochemical Analysis

For cross-sectional IHC analysis, samples were fixed on the insert membrane with 4% PFA for 15 min and paraffin embedded. Embedded samples were sectioned at 4 μm and stained for type II and type I collagen to detect mature chondrogenesis, MMP13 to detect hypertrophy, and laminin to detect adhesive molecules. Briefly, antigen retrieval was conducted by incubating with a 1:50 dilution of 50× Low pH Target Retrieval Solution (Dako, Agilent Technologies, CA, USA) for 15 min at 106-110° C. at low pressure in a pressure cooker for type II collagen samples and with Proteinase K (Dako, Agilent Technologies) for 6 min at room temperature (RT) for type I collagen, MMP13 and laminin samples. Laminin samples were permeabilized with 0.1% Triton X-100 (Sigma-Aldrich) for 10 min at RT. Non-specific binding was blocked for type II collagen, MMP13, and laminin samples with 10% Normal Goat Serum (Vector Laboratories, CA, USA) and with 5% Normal Donkey Serum (Abcam) for type I collagen samples, at RT for 30 min. Type II collagen, type I collagen, MMP13, and laminin samples were then incubated in a 1:200 dilution of Anti-Collagen Type II primary antibody (Thermo Fisher Scientific), a 1:200 dilution of Anti-Collagen Type I primary antibody (Thermo Fisher Scientific), a 1:200 dilution of Anti-MMP13 primary antibody (Abcam), or a 1:100 dilution of Anti-laminin primary antibody (Abcam) at 4° C. overnight, respectively. Samples were washed with 1×PBS and incubated in either a 1:200 dilution of Goat Anti-Rabbit 488 secondary antibody (Thermo Fisher Scientific) for type II collagen, MMP13, and laminin samples or a 1:200 dilution of Goat Anti-Donkey 594 secondary antibody (Thermo Fisher Scientific) for type I collagen samples at RT, covered, for 2 h. After 2 hours, samples were washed with 1×PBS and mounted with a DAPI-containing mounting medium (Invitrogen, MA, USA). Samples were visualized with a Zeiss Axio widefield microscope and ZEN software. Samples were visualized with a BX 41 widefield microscope using AmScope Software. To determine cytoskeletal arrangement, phalloidin (F-actin) staining was conducted. Briefly, samples were permeabilized with 0.1% Triton X-100 (Sigma-Aldrich) for 15 min and washed with 1×PBS. Samples were then incubated with a 1:100 dilution of phalloidin AlexaFluor 488 (Life Technologies) at RT, covered, for 30 min. Samples were then washed with 1×PBS and incubated with DAPI solution (2 drops/mL, Life Technologies) at RT for 5 min. Samples were then washed with 1×PBS and prepared for mounting. Samples were imaged with a confocal microscope (Nikon A1—NIS Elements AR Software). Images were prepared using ImageJ software.

1.5. Real-Time Quantitative PCR Analysis

RNA from samples was extracted using 1 mL TRIzol/sample (Ambion, Life Technologies, CA, USA) with a pestle motor mixer. Total RNA was isolated with the PureLink RNA Mini Kit (Invitrogen, Thermo Fisher Scientific) according to manufacturer instructions. For cDNA synthesis, all comparative samples were synthesized at the same time. Before synthesizing cDNA, the RNA was quantified with a NanoDrop Spectrophotometer (Thermo Scientific, USA), and all cDNA samples were prepared from 1 μg of RNA/sample. All samples with a purity (A₂₆₀/A₂₈₀) greater than 1.8 were deemed pure enough to use. cDNA synthesis was conducted using a High Capacity cDNA Reverse Transcriptase Kit (Applied Biosystems, Thermo Fisher Scientific, MA, USA) as per manufacturer instructions. Real-time qPCR analysis was conducted with TaqMan Universal PCR Master Mix (Applied Biosystems, Thermo Fisher Scientific) using an Applied Biosystems Step-OnePlus instrument. Gene expression levels were analyzed for the following genes: 1) glyceraldehyde 3-phosphate dehydrogenase (GAPDH, Hs99999905_m1) as a housekeeping gene, 2) β-actin (Hs99999903_m1), 3) β-catenin (Hs00355049_m1), 4) bone morphogenic protein 2 (BMP2, Hs00154192_m1), 5) cartilage oligomeric matrix protein (COMP, Hs00164359_m1), 6) SRY-box 9 (SOX9, Hs01001343_g1), 7) aggrecan (ACAN, Hs00153936_m1), 8) collagen type II alpha 1 chain (COL2A1, Hs00264051_m1), 9) collagen type I alpha 1 chain (COL1A1, Hs00164004_m1), 10) collagen type X alpha 1 chain (COLX, Hs00166657_m1), 11) matrix metallopeptidase 13 (MMP13, Hs00942584_m1). All primers were manufactured by Applied Biosystems. Relative gene expression was calculated by the quantitative comparative CT method. Gene expression was normalized to GAPDH expression levels. For cytoskeletal analysis and time comparison differentiation, expression levels are relative to the 0-day 2D monolayer control group, and for chondrogenic differentiation, expression levels are relative to the 2D monolayer 3-week control group.

1.6. Post-Differentiation Manipulation and Re-Attachment

For assessing structural changes during post-differentiation manipulation and adhesion capabilities, 3-week chondrogenically differentiated contracted hBM-MSC sheets were cut in half using a scalpel. Half of each sheet was immediately fixed in 4% PFA for 15 min and paraffin embedded. The other half of each sheet was re-plated onto FBS-coated 35 mm tissue culture plastic dishes. To transfer the cell sheets, cell sheet halves were nudged off cell culture insert membranes and manually transferred to new FBS-coated culture dishes with forceps. After placement on the secondary surface, sheets were incubated in chondrogenic medium for 1 hour to attach. After 1 hour, fresh chondrogenic media was added and cell sheets were moved to a hypoxia incubator for further culture. Brightfield images were captured of cells at sheet edges between 0 and 72 hours during the 3-day culture period with a Zeiss Axio widefield microscope and ZEN software. After 3 days, the cell sheet halves were fixed in 4% PFA for 15 minutes and paraffin embedded. For comparison, 3-week chondrogenically differentiated pellets were also re-plated onto FBS-coated 35 mm tissue culture plastic dishes. Pellets were manually transferred to the new culture surfaces by gentle pipetting. After placement on the secondary surface, pellets were incubated in a small amount of chondrogenic medium for 1 hour to attach. After 1 hour, fresh chondrogenic media was added and pellets were moved to a hypoxic incubator and cultured for an additional 6 hours. Construct attachment efficacy was quantified as a ratio of attached sheets or pellets after this additional 6 hours of culture to total number of sheets or pellets transferred (n=6).

1.7. Post-Differentiation Transplantation Potential

To determine transplantation potential to target cartilage tissue, 3-week chondrogenically differentiated contracted sheets were cut in quarters using a scalpel. One quarter of each sheet was immediately fixed in 4% PFA for 15 min and paraffin embedded. The other quarters of each sheet were transplanted onto the apical side of fresh (same day as harvest) ex vivo human articular cartilage pieces (˜2 cm²) harvested as de-identified tissue discards from human hip articular cartilage during routine hip arthroscopy procedures. After 3 days co-culture in a hypoxia incubator, the cell sheet-on-cartilage samples were fixed in 4% PFA for 3 days and paraffin embedded.

1.8. Statistical Analysis

All statistical analysis was completed on data sets of n≥4 biological replicates. All quantitative values are expressed as a mean±standard deviation. Statistical significance was evaluated using 2-tailed, paired, Student's t-tests assuming normal distribution of data. Sufficient normal distribution for this statistical analysis was defined as skewness of data≥−0.8 and ≤+0.8. Statistical significance was defined as not significant (ns) p≥0.05, *p<0.05, and **p<0.01.

2. Results

2.1. Spontaneous Post-Detachment Contraction of hBM-MSC Sheets

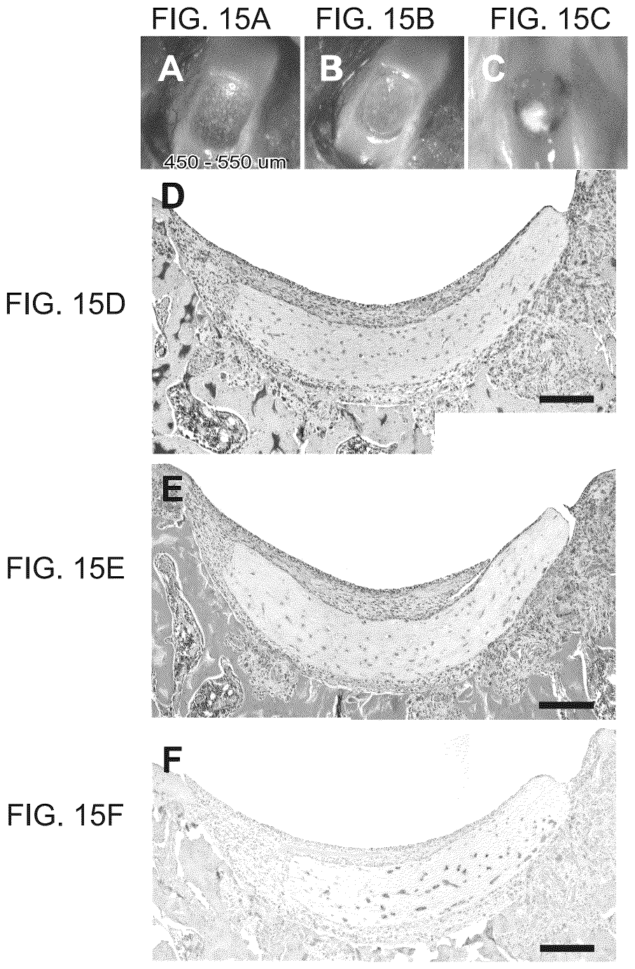

Cultured human bone marrow-derived mesenchymal stem cell (hBM-MSC) sheets spontaneously contracted after detachment from temperature-responsive culture dishes (FIG. 15). Spontaneous post-detachment contraction considerably altered the size and structure of the resulting 3D contracted cell sheet compared to the 2D monolayer cell culture conditions (FIG. 15). hBM-MSC sheets showed significant reductions in diameter (3.5-fold decrease, p=1.77E-5) (FIG. 15) and increased thickness (7-fold increase, p=3.63E-6) (FIG. 15) after post-detachment cell sheet contraction compared to 2D monolayer cell culture conditions.

2.2. Sheet Cytoskeletal Changes for Chondrogenic Potential

Cultured hBM-MSC sheet cytoskeletal arrangements were observed with phalloidin (F-actin) fluorescent staining, and nuclei were identified by DAPI (FIG. 21a,b). At day 0, cells within 2D cultures exhibited elongated and aligned cytoskeletal structures associated with standard adherent culture of MSCs (FIG. 10A). Conversely, harvested hBM-MSC cell sheets allowed to contract after detachment exhibited a more random, crossed-fiber structure with more rounded nuclei (FIG. 10B). These cytoskeletal changes were confirmed as significant based on relative β-actin gene expression (FIG. 10C) between contracted 3D sheets and 2D monolayer cultures (p=0.0194). In addition to these cytoskeletal changes, contracted 3D sheets showed significant increases in cell-cell interactions, β-catenin (p=0.00865), and pro-chondrogenic signaling molecules, BMP2 (p=0.0000457) and COMP (p=0.000947), (FIG. 10D-F) compared to 2D monolayer cultures prior to chondrogenic induction.

2.3. Increased Chondrogenic Potential of hBM-MSCs as 3D Contracted Sheets

hBM-MSCs were chosen as an MSC source with documented chondrogenic potential, which was confirmed in standard 3D pellet cultures (see FIG. 2). 3-week chondrogenic differentiation of hBM-MSCs harvested as 3D contracted cell sheets resulted in positive hyaline-like chondrogenesis (FIG. 11). Positive Safranin-O and type II collagen staining were identified in 3-week differentiated samples compared to control samples (FIG. 11 a-d, g-j). Safranin-O stains sulfated proteoglycans red (depth of red color is relative to GAG content) with Fast Green counterstaining other ECM proteins blue. Type II collagen is denoted by red fluorescence (pseudo red immunostaining) and nuclei are counterstained with DAPI (blue). Monolayer 2D hBM-MSC cultures exhibited some slightly positive chondrogenic staining with Safranin-O (FIG. 11G) and type II collagen (FIG. 11I) after 3-week differentiation. The 3D contracted sheets after 3-week differentiation stained more intensely for all chondrogenic markers (Safranin-O (FIG. 11H) and type II collagen (FIG. 11J)) compared to the chondrogenic 2D cultures (FIG. 11G,I). The differentiated 3D contracted sheets also developed lacunae structures associated with mature hyaline cartilage (FIG. 11H). After 3 weeks of differentiation, most cells in the 3-week differentiated contracted sheets exhibited condensed, rounded cytoskeletal structures associated with mature chondrocytes (FIG. 11N). Some cells in 2D monolayer cultures displayed this rounded cytoskeletal structure; however, the majority of cells in the 3-week differentiated monolayers had more variable spindle-like or fibroblastic cell shapes (FIG. 11M). Gene expression of all chondrogenic markers were significantly increased (SOX9 (FIG. 11Q) (p=0.0044), ACAN (FIG. 11R) (p=0.0242), and COL2A1 (FIG. 11S) (p=0.0126)) in 3D contracted cell sheets compared to 2D cell cultures after 3 weeks of differentiation. 3D contracted cell sheets also expressed significantly higher ratios of type II to type I collagen (FIG. 11T) (p=0.0107) with minimal staining for type I collagen (FIG. 11L) compared to 2D cultures (FIG. 11K) after 3 weeks of differentiation. Additionally, 3D contracted sheets showed a 30-fold increase in thickness after 3 weeks of differentiation, significantly greater than the 23-fold increase in 2D cell culture thickness (FIG. 11O) (p=4.26E-6). This increase in thickness after differentiation did not significantly change the number of cells within either the 3D contracted cell sheets or 2D cell cultures (FIG. 11P) (p=0.422 (3D); 0.997 (2D)), suggesting that increased thickness of chondrogenic cell sheets results from chondrogenically induced ECM deposition.

2.4. Chondrogenic Differentiation of Cell Sheets Over Time

Chondrogenic differentiation of 3D contracted harvested cell sheets and pellets over time showed very similar progressions in chondrogenesis, with slightly earlier onset of sulfated GAG accumulation and delayed onset of hypertrophic and fibrocartilage phenotypes seen in 3D contracted hBM-MSC sheets (FIG. 12). Both pellet cultures and 3D sheets were negative for Safranin-O staining at day-0 (FIG. 12A,E). After 1-week differentiation, 3D contracted cell sheets (FIG. 12F) showed greater Safranin-O staining throughout the sample than pellet cultures (FIG. 12B), but staining was faint for both samples. By 3-week differentiation, both 3D cell sheets (FIG. 12H) and pellet cultures (FIG. 12D) stained strongly for Safranin-O (FIG. 12H,D) and type II collagen (FIG. 12Q), while exhibiting lacunae structures. Gene analysis showed a similar trend for chondrogenic marker expression (SOX9, COL2A1, ACAN) over 3-weeks of differentiation for contracted sheets and pellets (FIG. 12S). Chondrogenic marker expression was not significantly different between the 3D sheets and pellet cultures at any time point during differentiation (Sox 9: p=0.435 (0 day), 0.754 (1 W), 0.189 (2 W), 0.222 (3 W); COL2A1: p=0.169 (0 day), 0.158 (1 W), 0.265 (2 W), 0.131 (3 W); ACAN: p=0.773 (0 day), 0.681 (1 W), 0.213 (2 W), 0.724 (3 W)).

Although chondrogenic expressions were similar, 3D contracted sheets exhibited delayed onset of hypertrophic and fibrocartilage characteristics compared to standard pellet cultures. Immunohistochemical staining for hypertrophic marker, MMP13,^{44,45} was negative in day-0 pellet cultures and 3D sheets (FIG. 12I,M). MMP13 staining in hBM-MSC pellet cultures remained negative or minimal through 2 weeks (FIG. 12I-K), but was highly expressed in 3-week samples (FIG. 12L). MMP13 staining remained negative or minimal in 3D sheet samples through 3 weeks (FIG. 12M-P). By 3-week differentiation, pellet cultures were stained strongly for fibrocartilage marker type I collagen, whereas 3D contracted sheets showed minimal type I collagen positive staining (FIG. 12R). Hypertrophic type X collagen gene expression increased throughout chondrogenic differentiation for both 3D sheets and pellets (FIG. 12T); however, expression of type X collagen was significantly higher in pellet culture than in 3D sheets after 3-weeks of differentiation (p=0.00596). MMP13 gene expression was low during early chondrogenesis for both constructs, but significantly higher in pellets compared to 3D contracted cell sheets at 3 weeks (FIG. 12T) (p=0.0114). Expression of cell-cell adhesion marker β-catenin, which is related to chondrogenic commitment during early stages of chondrogenesis, but is also associated with chondrocyte hypertrophy if overexpressed during late stages of chondrogenic differentiation, was significantly higher in 3D cell sheets at the time of induction, but then significantly lower throughout differentiation compared to hBM-MSC pellet cultures (FIG. 12U) (p=0.00248 (0 day); 0.0374 (1 W); 0.00591 (2 W); 0.0464 (3 W)).

2.5. Hyaline-Like Cell Sheet Manipulation without Affecting Sheet Characteristics

3D contracted hBM-MSC sheets differentiated for 3 weeks were able to be manipulated and transferred as intact sheets to new culture surfaces (FIG. 13). After 3 days of secondary culture on FBS-coated surfaces, cell sheets were harvested, fixed and stained with Safranin-O. This staining showed no discernable changes to the structure or GAG composition of the cell sheets after transfer (FIG. 13A,B). During secondary culture, cell migration/proliferation was observed at edges of the cell sheets as early as 6 hours after transferring the harvested, differentiated, 3D contracted sheets (FIG. 13C), indicating rapid, spontaneous surface adhesion and cell viability. Compared to 100% attachment success rate for the 3-week differentiated cell sheets (6/6 sheets adhered completely), none of the re-plated 3-week differentiated pellets were able to adhere and remain attached to the secondary culture dish (0/6 pellets adhered) after fresh media was added and constructs were cultured for an additional 6 hours (FIG. 13D). Immunohistochemical analysis of the 3-week differentiated 3D contracted sheets and pellets showed that adhesion molecule laminin staining was strongly expressed along the cell sheet basal side (FIG. 13E), whereas laminin staining of the pellet cultures showed minimal positive staining around the peripheral of the pellet (the interface surface) (FIG. 13F).

2.6. Hyaline-Like Cell Sheet Transplantation Potential

3D contracted hBM-MSC sheets differentiated for 3 weeks were able to spontaneously and strongly adhere to fresh, ex vivo, human articular cartilage pieces (FIG. 14). Post-differentiation 3D hBM-MSC sheets (FIG. 14A) strongly adhered to fresh, ex vivo, human articular cartilage surfaces within 1 hour (FIG. 14B) and remained attached for at least 3 days in continued culture. Safranin-O staining after 3-days of co-culture showed close physical adhesion between the sheet and the cartilage surface, with few to no gaps seen along the interface (FIG. 14C). Laminin staining after 3-days of co-culture was most intense at the interface between the sheet and the cartilage surface (FIG. 13E), supporting continued adhesion and possible biological binding between cell sheets and target cartilage tissue.

3. Discussion

Many studies demonstrate that MSCs exhibit increased chondrogenic potential in 3D structures compared to 2D structures. Unlike 2D cell cultures, 3D cultures allow cells to assume rounded cell morphologies associated with chondrocyte cytoskeletal organization. The contracted cell sheets spontaneously produce this 3D environment for cells to assume a more rounded and less elongated cytoskeletal structure, which is directly related to their chondrogenic potential. The cytoskeletal reorganization and transition from 2D to 3D culture seen in contracted cell sheets upon temperature-mediated detachment (FIGS. 1 and 10) are most likely caused by changes in stromal cell tensegrity, where cell release from anchored/adherent culture allows spontaneous contraction of actin filaments, prompting contraction of cells within cell sheets.

Cell sheet technology spontaneously detaches cells as confluent sheets without harvesting enzymes or damage to the endogenous cell-cell and cell-ECM interactions, maintaining endogenous cellular contractile forces of these collective interactions along actin filaments, which stimulates sheet contraction as a contiguous unit. This post-detachment cell sheet contraction spontaneously fabricates 3D, multi-nuclei thick, scaffold-free cell sheet structures (FIG. 1) and induces cytoskeletal reorganization (FIG. 10). These changes in cytoskeletal structure may also stimulate mechanotransduction, mimicking early chondrogenic condensation by changing both cell shape and ECM structure, resulting in increases in cell-cell interactions via β-catenin and upregulation of pro-chondrogenic signaling molecules BMP2 (regulator of cellular condensation) and COMP (regulator of collagen accumulation and ECM assembly) prior to chondrogenic induction.

Structural transitions and upregulation of pro-chondrogenic signaling prior to chondrogenic induction results in 3D contracted cell sheets achieving chondrogenic phenotypes after induction with chondrogenic medium (FIG. 11). Healthy articular cartilage specifically has hyaline chondrogenic phenotypes, including ECM rich in type II collagen and proteoglycans (which enable resistance to shear, compressive, and tensile forces), expression of SOX9 and aggrecan, low expression of type I collagen, nuclei in lacunae structures, and low cellular densities relative to ECM. The harvested 3D contracted sheets successfully achieve these standard and accepted benchmarks of hyaline-like phenotypes after differentiation: significant type II collagen and proteoglycan content in the ECM, high expression of common hyaline cartilage markers (SOX9, COL2A1, ACAN), low expression of type I collagen with a high COL2A1/COL1A1 ratio, and rounded cell structures with nuclei in lacunae structures at relatively low cellular densities. In addition to ECM composition (i.e. proteoglycan, aggrecan, and type II collagen content), ECM deposition (as seen in differentiating 3D contracted sheets) is also associated with hyaline chondrogenic differentiation. The cytoskeletal reorganization within 3D contracted cell sheets prior to chondrogenic induction upregulates COMP and BMP2, which are directly associated with ECM assembly and collagen accumulation, resulting in significantly more ECM deposition in the 3D cell sheets than in the 2D cultures after chondrogenic induction. 3D cell sheets, generated from spontaneous contraction upon temperature-mediated detachment from culture surfaces, are initially cell-dense structures; however, ECM deposition that significantly increases 3D cell sheet thickness during chondrogenesis decreases the construct's overall cellular density. This reduction in overall cellular density from ECM deposition results in a hyaline-like tissue construct that more closely matches native hyaline cartilage structure and cellular distribution.

A major limitation of MSC chondrogenic differentiation to hyaline-like phenotypes is the inevitable progression of MSC-derived chondrocytes towards hypertrophy and fibrocartilage both in vitro and in vivo. Although 3D structures are clearly necessary for proper hyaline-like chondrogenic differentiation, specific thresholds must be determined as construct thickness and cellular densities have been shown to impact media diffusion, affecting chondrogenic differentiation and hypertrophy by creating areas of low oxygen tension and increasing nutrient diffusion gradients in thicker tissues. In this study, 3D cell sheets exhibited a similar progression of chondrogenic development, but a delayed onset of hypertrophic characteristics compared to control 3D pellet cultures in vitro (FIG. 12). Although the driving mechanism of MSC-derived chondrocyte hypertrophy in vitro is still largely unknown, the observed delayed onset of MSC sheets' hypertrophic characteristics in the present study most likely results from the sheets' structural characteristics. Specifically, 3D cell sheets used in this study are thinner, with reduced cellular densities, compared to control 3D pellet cultures, promoting more substantial and uniform media diffusion throughout the construct, allowing chondrogenesis to be primarily driven by media supplementation rather than cellular signaling. Continued over-expression of cellular signaling, such as β-catenin, during chondrogenic differentiation has been linked to inducing chondrocyte hypertrophy in vitro via activation of the canonical Wnt pathway. The 3D pellet cultures' greater thickness, combined with higher cellular densities, present a barrier to sufficient nutrient diffusion, which may increase reliance on these cell-cell interactions (i.e. β-catenin upregulation (FIG. 12) to propagate chondrogenic cues to cells at the pellets' cores poorly exposed to free media. These data together suggest that tailoring construct thickness and cellular densities of 3D cell sheets may modulate cellular interactions during chondrogenesis, delaying the onset of MSC-derived chondrocyte hypertrophy in vitro. It is important to assess and monitor any transitions toward hypertrophic or fibrocartilage phenotypes during in vitro differentiation as these phenotypes would be detrimental to enacting prolonged therapeutic benefit in future in vivo studies requiring stable hyaline-like tissue. Further in vitro studies are necessary to elucidate optimal construct thicknesses and specific mechanisms driving this hypertrophic transition and identify the most effective sheet preparation parameters for producing hyaline cartilage in vitro while preventing MSC-derived chondrocyte hypertrophy.

Various differentiation platforms have successfully promoted chondrogenic differentiation of MSCs to hyaline-like phenotypes in vitro; however, none of these differentiation products has been successfully translated to human applications. For example, pellet cultures are used primarily for in vitro verification of differentiation potential rather than in vivo therapeutic applications, based on limitations in adhesion capabilities and homogeneity of regenerated tissue. Clusters of pellet cultures have been used to fill cartilage defects in vivo, and have shown some capacity to populate the negative space left by the pellets' spherical shape constraints. However, these pellet clusters do not create homogenous tissues and do not strongly adhere to biologic surfaces without additional glues or support membranes to contain them at defect sites.

One unique benefit of cell sheet technology is the ability to directly and spontaneously transplant cells without scaffolds or support materials to target tissue sites via retention of endogenous ECM, cell interactions, and intact adhesion proteins, which also provide a stable cell culture environment for interacting with the native tissues. Our data show that cell sheets can be transferred after differentiation, adhere to biologic surfaces, and that this transfer does not affect the structure or chondrogenic characteristics (FIG. 13). Maintenance of sheet chondrogenic and structural characteristics after manipulation and transfer is promising for rapidly replacing damaged or missing hyaline cartilage. These differentiated cell sheets were also able to adhere strongly to fresh ex vivo human cartilage and potentially begin interfacing with native chondrocytes within 3 days (FIG. 13). These endogenous adhesion capabilities are attributed to retention of adhesion molecules along the sheets' basal surface post-differentiation, which are not abundant along the periphery of the pellet cultures, likely causing the disparity in transplantation potential. Retention of surface adhesion molecules is expected to aid in cell sheet engraftment and localization at cartilage defect sites. Close physical and biochemical interfacing between hyaline-like cell sheets and native cartilage is expected to help maintain sheets' hyaline characteristics in vivo via direct chondrogenic signaling from the host cartilage. This ex vivo experiment represents an ideal interaction between the cell sheets and healthy articular cartilage in the absence of all other defect microenvironmental factors. To fully verify sheet transplantation capabilities and therapeutic benefits for chondrogenically differentiated cell sheets in articular cartilage focal defects, in vivo testing with long-term follow-ups and specific focus on regeneration and immune response will be necessary.

In this study, we demonstrate that 1) MSC sheets are able to chondrogenically differentiate to hyaline cartilage in vitro without scaffold materials after spontaneous post-detachment cell sheet contraction via structural transformation and cytoskeletal reorganization, 2) these 3D MSC sheets provide a suitable initial thickness and cellular density that delays hypertrophy while maintaining hyaline-like chondrogenic phenotypes in vitro, and 3) after differentiation, these 3D cell sheets can spontaneously adhere directly to cartilage surfaces, maintaining their structural and chondrogenic characteristics and potentially interfacing with the native tissue via retained adhesion proteins. Based on these findings, 3D MSC sheets represent a distinct platform for developing allogeneic, transplantable, scaffold-free hyaline cartilage constructs for articular cartilage regeneration therapies.

4. Conclusions

Cell sheet-based technology presented in this study represents an improved strategy for fabricating scaffold-free cartilage constructs with hyaline-like characteristics in vitro. Furthermore, hyaline-like chondrogenically differentiated 3D MSC sheets spontaneously adhere and begin interfacing with cartilage tissue without damaging structural or chondrogenic characteristics. Our cell sheet-based technique using MSCs should provide an adaptable platform to generate transplantable hyaline-like constructs in vitro to rapidly and directly replace damaged hyaline articular cartilage.

### Example 3

The transplantation ability of the chondrogenically differentiated cell sheets described in Examples 1 and 2 was evaluated in vivo in a rat model of focal cartilage defects. A 2 mm focal defect was made in a nude (RNU) rat hind leg trochlear groove (see FIG. 15A), where successful defect creation removes the full thickness of cartilage without disrupting the subchondral bone (little to no bleeding is seen from the subchondral bone). A 3-week chondrogenically differentiated hyaline-like human MSC-derived cell sheet was transplanted to the defect (see FIG. 15B) at the time of defect creation. Sheets were allowed to adhere for about 30 min in a slightly humidified environment before the joint was closed. Two weeks post-transplantation, the knee joint was harvested (FIG. 15C) to assess cell sheet transplantation capacity. Cross-sectional histological analysis of the 3-week differentiated human cell sheet in the rat focal defect at 2 weeks post-transplantation was performed with Safranin-O (FIG. 15D), Hematoxylin & Eosin (FIG. 26E), and human-specific vimentin immunohistochemical staining (FIG. 15F). Positive Safranin-O staining showed retention of positive hyaline-like characteristics of the cell sheet in the transplant area (FIG. 15D). H&E staining indicated integration with the host tissue (FIG. 15E). Vimentin is a type III intermediate filament protein that is expressed in mesenchymal-derived cells, and therefore human-specific vimentin can be used to specifically identify human cells within a xenogeneic host. Transplanted human cells within the cell sheet were positive for human-specific vimentin indicating engraftment and retention in the transplant area (FIG. 15F). Scale bars=200 μm. Overall, these results indicate that chondrogenically differentiated hyaline-like human MSC-derived cell sheets may be successfully transplanted to cartilage and integrated with host tissue while maintaining hyaline-like characteristics.

We claim:

1. A chondrogenic cell sheet comprising at least two layers of confluent chondrogenically differentiated cells, wherein the cell sheet is prepared from mesenchymal stem cells (MSCs), and wherein the chondrogenically differentiated cells on the basal side of the cell sheet express one or more adhesion molecules.

2. The chondrogenic cell sheet of claim 1, wherein the MSCs are human bone marrow MSCs (hBM-MSCs).

3. The chondrogenic cell sheet of claim 1, wherein the cell sheet consists essentially of chondrogenically differentiated cells.

4. (canceled)

5. The chondrogenic cell sheet of claim 1, wherein the cell sheet comprises an extracellular matrix.

6. The chondrogenic cell sheet of claim 5, wherein the extracellular matrix comprises a protein selected from the group consisting of type II collagen and a sulphated proteoglycan.

7. The chondrogenic cell sheet of claim 5, wherein the chondrogenically differentiated cells express a protein selected from the group consisting of SOX9, aggrecan, COL2A1, ACAN, COMP and BMP2.

8. The chondrogenic cell sheet of claim 1, wherein the cell sheet comprises lacunae structures.

9. The chondrogenic cell sheet of claim 1, wherein the one or more adhesion molecules are selected from fibronectin and laminin.

10. The chondrogenic cell sheet of claim 1, wherein the cell sheet exhibits physical adhesion to a cartilage surface after transplantation to the cartilage surface.

11. A method of preparing a chondrogenic cell sheet comprising:
a) culturing mesenchymal stem cells (MSCs) on temperature responsive cultureware until confluent to form a cell sheet;
b) detaching the cell sheet by temperature reduction and allowing the cell sheet to contract, forming a contracted cell sheet;
c) contacting the contracted cell sheet with a culture surface; and
d) treating the contracted cell sheet on the culture surface with chondrogenic medium and culturing to form a chondrogenic cell sheet.

12. The method of claim 11, wherein the contracted cell sheet is grown on the culture surface for at least 3 weeks.

13. The method of claim 11, wherein the chondrogenic medium comprises a protein selected from the group consisting of transforming growth factor beta (TGFβ) and a bone morphogenic protein (BMP).

14. The method of claim 11, wherein the cell sheet in step b) is detached from the temperature responsive cultureware without treating the cell sheet with one or more enzymes.

15. A chondrogenic cell sheet produced by the method of claim 11.

16. A method of transplanting a chondrogenic cell sheet to a subject in need thereof, comprising applying the chondrogenic cell sheet of claim 1 to a tissue of a subject.

17-18. (canceled)

19. A method of repairing cartilage tissue in a subject in need thereof, comprising applying the chondrogenic cell sheet of claim 1 to cartilage of the subject, thereby repairing the cartilage tissue in the subject.

20-24. (canceled)

25. A method of treating or preventing a joint disease in a subject in need thereof, comprising applying the chondrogenic cell sheet of claim 1 to a joint in the subject, thereby treating or preventing the joint disease in the subject.

26-29. (canceled)

30. A method of preventing osteoarthritis in a subject having a symptomatic cartilage defect caused by acute or repetitive trauma, the method comprising applying the chondrogenic cell sheet of claim 1 to the cartilage having the defect, thereby preventing the osteoarthritis in the subject.

31. (canceled)

32. The method of claim 16, wherein the chondrogenically differentiated cells in the cell sheet are allogeneic to the subject.

33. The method of claim 16, wherein the chondrogenically differentiated cells in the cell sheet are autologous to the subject.
